SEQUENCE LISTING (2 sequences)

SEQ ID NO: 1 - DNA, 20 bp, Homo Sapiens
tcgtcacctcttctgccttt

SEQ ID NO: 2 - DNA, 20 bp, Homo Sapiens
acacaccatagccgtcatca

(19) United States
(12) Patent Application Publication
(10) Pub. No.: US 2017/0304251 A1
(43) Pub. Date: Oct. 26, 2017

(54) AVOCADO-DERIVED LIPIDS FOR USE IN TREATING LEUKEMIA

(71) Applicants: Paul Anthony SPAGNUOLO, Oakville (CA); Aaron David SCHIMMER, Thornhill (CA)

(72) Inventors: Paul Anthony Spagnuolo, Oakville (CA); Aaron David Schimmer, Thornhill (CA); Eric Alexander Lee, Waterloo (CA)

(21) Appl. No.: 15/517,914

(22) PCT Filed: Oct. 9, 2015
(86) PCT No.: PCT/CA2015/051027

§ 371 Date: Apr. 7, 2017

Related U.S. Application Data
(60) Provisional application No. 62/061,892, filed on Oct. 9, 2014.

(51) Int. Cl. A61K 31/22; A61K 31/08; A61K 31/047; C07C 33/042; G01N 33/50; G01N 33/574

(52) U.S. Cl. CPC A61K 31/22; G01N 33/5014; G01N 33/57426; A61K 31/047; C07C 33/042; A61K 31/08; G01N 2800/50

(57) ABSTRACT

A method of treating a leukemia comprising administering to a subject in need thereof a therapeutically effective amount of a composition comprising a compound of Formula (I) and/or (II) having the structure: OR2 R R1O n OR2 R R1O n I II wherein: 10 ---- represents a single or a double bond; R is OH when C----R is C—R, and R is O when C----R is C═R; n is 1, 3, 5 or 7; and R1 and R2 are independently hydrogen or acetyl, and/or isomers, stereoisomers or solvates thereof and/or mixtures thereof.

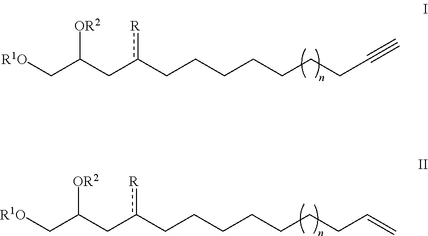

### RELATED APPLICATION

This Patent Cooperation Treaty application claims priority to United States Provisional Patent application having Ser. No. 62/061,892 filed on Oct. 11, 2014, which is incorporated by reference herein in its entirety.

### FIELD

The disclosure relates to methods and compositions for the treatment of leukemia and particularly to methods and compositions comprising an avocado-derived lipid compound such as avocadyne, avocadyne acetate, avocatin A and avocatin B optionally in combination with a chemotherapeutic for the treatment of leukemia such as acute myeloid leukemia.

### BACKGROUND

Leukemia and leukemia stem cells (LSCs) possess several mitochondrial features that distinguish them from normal hematopoietic cells. Compared to normal cells, leukemia cells contain greater mitochondrial mass and have higher rates of oxidative phosphorylation¹ and fatty acid oxidation².

Avocatin compounds, which are polyhydroxylated fatty alcohols, are compounds extracted from avocado pear seeds and identified by Alves et al in 1970²⁰. Avocatins are a class of natural products with known cosmetic and therapeutic applications. For example, U.S. Pat. No. 6,582,688 describes a method for extracting furan lipids and polyhydroxylated fatty alcohols from avocados, and therapeutic, cosmetical or food uses thereof. U.S. application Ser. No. 11/597,634 describes a method for preventing and/or treating obesity comprising administering alkylfurans. U.S. application Ser. No. 13/062,758 teaches a method for treating a subject with an inflammatory disease, the method comprising administering a pharmaceutical composition comprising polyhydroxylated fatty alcohols.

Acute myeloid leukemia (AML) is a devastating disease characterized by the accumulation of malignant myeloid precursors (i.e., blasts) that fail to terminally differentiate³. Patients diagnosed with AML are faced with poor disease prognosis. In adults (>65), 2-year survival rates are less than 10%⁴. The suboptimal quality of current therapy is, in part, attributed to the inability of current drugs to target and eliminate LSCs. Thus, new therapeutic strategies that target both the bulk and LSC populations are needed to improve AML outcomes.

### SUMMARY

Accordingly, an aspect of the disclosure is a method of treating a leukemia comprising administering to a subject in need thereof a therapeutically effective amount of a compound of Formula (I) and/or (II) having the structure:

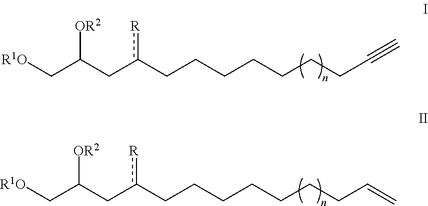

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers, or solvates thereof and/or mixtures thereof.

In an embodiment, the compound of Formula (I) and/or (II) and/or isomers, stereoisomers, or solvates thereof and/or mixtures thereof is a compound that decreases mitochondrial fatty acid oxidation in a leukemia cell.

In another embodiment, the compound of Formula (I) and/or (II) and/or isomers, stereoisomers, or solvates thereof and/or mixtures thereof is a compound that decreases production of nicotinamide adenine dinucleotide hydrogen (NADH), nicotinamide adenine dinucleotide phosphate (NADPH) or glutathione (GSH) in a leukemia cell by for example at least 30%, by at least 40%, by at least 50%, or by at least 60% compared to an untreated leukemia cell.

Another aspect of the disclosure is a combination comprising a compound of Formula (I) and/or (II) and/or isomers, stereoisomers, or solvates thereof and/or mixtures thereof and a chemotherapeutic.

In another aspect, the combination comprising a compound of Formula (I) and/or (II) and/or isomers, stereoisomers, or solvates thereof and/or mixtures thereof and a chemotherapeutic are for use in the treatment of a leukemia in a subject in need thereof.

In an embodiment, the chemotherapeutic is cytarabine. In another embodiment, the chemotherapeutic is an anthracycline compound such as daunorubicin, doxorubicin, mitoxantrone, idarubicin and amsacrine.

In yet another embodiment, the leukemia is selected from acute myeloid leukemia (AML), acute lymphocytic leukemia (ALL), chronic lymphocytic leukemia (CLL) and chronic myelogenous leukemia (CML).

In an embodiment, the pharmaceutical composition is for a use, method, combination or kit described herein and comprises a therapeutically effective amount of a compound of Formula (I) and/or (II) and/or isomers, stereoisomers, or solvates thereof and/or mixtures thereof and one or more suitable excipients, diluents, buffers, carriers or vehicles.

In another embodiment, the pharmaceutical composition is for use in treating leukemia in a subject in need thereof.

Another aspect of the disclosure is a test assay for identifying putative combinations for treating leukemia comprising:
a. contacting a leukemia cell with a compound of Formula (I) and/or (II) and/or isomers, stereoisomers, salts or solvates thereof and/or mixtures thereof in the presence and in the absence of a test agent;
b. measuring the viability of the cell in the presence of the test agent and in the absence of the test agent, optionally using a 3-(4,5-dimethylthiazol-2-yl)-5-(3-carboxymethoxyphenyl)-2-(4-sulfophenyl)-2H-tetrazolium inner salt (MTS) assay;
c. determining if the compound of Formula (I) and/or (II) and/or isomers, stereoisomers, or solvates thereof and/or mixtures thereof, in combination with the test agent exhibit synergistic cytotoxicity; and
d. optionally testing synergistic combinations is a second viability assay.

Yet another aspect relates to a method of identifying a subject with leukemia likely to benefit from administration of a compound of Formula (I) and/or (II) and/or isomers, stereoisomers, or solvates thereof and/or mixtures thereof and optionally a chemotherapeutic, comprising:
a. obtaining a test sample comprising leukemia cells from the subject;
b. determining a mitochondrial mass of the test sample; and
c. comparing the mitochondrial mass of the test sample to a mitochondrial mass of a control;
   wherein the subject is identified as likely to benefit from administration of the compound of Formula (I) and/or (II) and/or isomers, stereoisomers, or solvates thereof and/or mixtures thereof and optionally in combination with the chemotherapeutic, when the leukemia cells have an at least 2 fold increased mitochondrial mass compared to the control.

Another aspect of the disclosure is a kit comprising a compound of Formula (I) and/or (II) and/isomers, stereoisomers, or solvates thereof, and/or mixtures thereof and one or more reagents for carrying out a method or use described herein, for example for measuring at least a 2 fold increased mitochondrial mass compared to the control, optionally including a chemotherapeutic, and/or packaging instructions for use thereof.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

An embodiment of the present disclosure will now be described in relation to the drawings in which:

FIG. 1: Avocatin B is selectively toxic toward AML cells. (A) (Top panel) A screen of a natural health product library identified avocatin B as the most potent compound at reducing TEX leukemia cell viability. Cells were incubated with compounds for 72 hours and cell growth and viability were measured by the MTS assay. Arrow indicates avocatin B. (bottom panel) Avocatin B's structure²¹. (B) Avocatin B's activity was tested in peripheral blood stem cells (PBSCs; n=4) isolated from GCSF stimulated donors or cells isolated from AML patients (n=6). Primary cells were treated with increasing avocatin B concentrations for 72 hours and viability was measured by the Annexin V (ANN)/Propidium Iodide (PI) assay and flow cytometry. Data are presented as log 10 EC50 values. (C) Avocatin B was tested in combination with cytarabine and doxorubicin using Calcusyn software as detailed in the methods section. Experiments were performed twice in triplicate; representative figure shown. (D) (top panel) Primary AML (n=3) and normal (n=3) cells were cultured with avocatin B (3 μM) for 7-14 days and clonogenic growth was assessed by enumerating colonies (colony defined as >10 cells). Data are presented as % clonogenic growth compared to control±SEM, similar to previously described^{1,49}. Experiments were performed twice in triplicate. (bottom panel) AML cells from one patient were treated with avocatin B (3 μM) for 48 hours or a vehicle control and then intrafemorally injected into sublethally irradiated, CD122 treated, NOD/SCID mice (n=10/group). After 6 weeks, human AML cells (CD45⁺/CD19⁻/CD33⁺) in mouse bone marrow were detected by flow cytometry. **p<0.01; ***p<0.001.

FIG. 2: Avocatin B is the most active avocado lipid analogue. (A) TEX cells were treated with increasing concentrations of avocado lipid analogues. Avocatin B imparted the greatest reducing in TEX cell viability. Data are presented as mean percentage of live cells (MTS assay)±SD from representative experiments. Experiments were performed three times in triplicate.

FIG. 3: Avocatin B induces mitochondria-mediated apoptosis. (A) TEX cells were treated with 10 μM avocatin B for increasing duration and phosphatidylserine exposure in live cells (i.e., apoptotic phenotype; ANN⁺/PI⁻) and (B) DNA fragmentation were measured by flow cytometry. Data are presented as fold change in apoptotic phenotype and percent cells in sub G1 peak, respectively. (C) TEX cells were treated with 10 μM avocatin B for increasing duration and caspase 3&7 activation and (D) cleavage of PARP, a substrate of caspase 3, were measured by a commercially available activation assay and Western blotting, respectively. (E) TEX cells were treated with 10 μM avocatin B in the presence and absence of the pan caspase inhibitor Z-VAD-FMK (ZVAD) or the caspase-3 specific inhibitor Q-VD-OPh (QVD). Viability was measured after a 72 hour incubation period by the MTS assay. Data are presented as percent change in viability compared to controls±SD. (F) TEX cells were treated with 10 μM avocatin B for increasing duration and cytochrome c and AIF release were measured in cytoplasmic fractions by flow cytometry. Data are presented as percent of cells releasing cytochrome c or AIF±SD. All experiments were performed three times in triplicate, representative figures are shown. *p<0.05; **p<0.01;*** p<0.001.

FIG. 4: Kinetics of avocatin B-induced death. TEX cells were treated with 10 μM avocatin B and (A) apoptotic cells (ANN+/PI−) and (B) cell viability (ANN−/PI−) were measured by flow cytometry. Data are presented as mean percentage of apoptotic or live cells±SD. All experiments were performed three times in triplicate.

FIG. 5: Cell cycle analysis of avocatin B treated TEX cells. TEX cells were incubated with avocatin B (10 μM) over a 48 hour time course and cell cycle analysis was performed by propidium iodide staining and flow cytometry. Data are presented as percentage of cells per cell cycle phase±SD from representative experiments. All experiments were performed three times in triplicate. ***; p<0.001.

FIG. 6: Avocatin B inhibits fatty acid oxidation resulting in reduced NADPH and elevated ROS. (A) Illustration of fatty acid oxidation in mitochondria. Long chain fatty acids (LCFA) enter the mitochondria via CPT1 for fatty acid oxidation to yield acetyl-CoA. Acetyl-CoA enters the TCA cycle to generate NADPH, an enzymatic co-factor and antioxidant. (ME: malic enzyme; IDH: isocitrate dehydrogenase; α-KG: α-ketoglutarate). (B) Oxidation of exogenous fatty acids was assessed by measuring the oxygen consumption rate (OCR) in TEX cells treated with palmitate (175 μM), avocatin B (10 μM), avocatin B and palmitate or palmitate and etomoxir (100 μM). Arrows indicate the time when oligomycin, CCCP and antimycin/rotenone were added to the cells. Effects on fatty acid oxidation were measured with the Seahorse Bioanalyzer, as detailed in the methods section, and (C) quantified by peak area after oligomycin and CCCP treatment, as described by the manufacturer's protocol and detailed in the methods. Data are presented as percent OCR compared to palmitate treated cells±SD. (D) NADPH was measured in TEX cells (top; t=3-5 hours) or primary AML cells (n=3; t=24 hours, bottom; results for OCI-AML2 cells are shown in FIG. 6J using the commercially available Amplite™ Fluorimetric Assay following treatment with avocatin B (10 μM), palmitate (175 μM) or etomoxir (100 μM), according to the manufacturer's protocol. Data are presented as a percent NADPH compared to vehicle control treated cells±SD. (E) Reactive oxygen species (ROS) were measured in TEX cells (top) or primary AML cells (n=3, rbottom; results for OCI-AML2 cells are shown in FIG. 6J) treated with 10 μM avocatin B for increasing time by DHE and DCFH-DA by flow cytometry. Data are presented as percentage of cells with increased ROS levels, compared to vehicle control, ±SD from representative experiments. (F) TEX cells were treated with 10 μM avocatin B in the presence or absence of the anti-oxidants, N-acetyl cysteine (NAC) or α-tocopherol (α-Toc). Daunorubicin (DNR) was used as a negative control. Viability was measured by the ANN/PI assay and data are presented as mean percentage of viable cells (i.e., ANN⁻/PI⁻)±SD from representative experiments. All experiments were performed three times in triplicate, representative figures are shown. *p<0.05; **p<0.01; ***p<0.001; (G), NADH was measured in TEX cells (top; t=3-5 hours) or primary AML cells (bottom n=3; t=24 hours; results for OCI-AML2 cells are shown in 6J) using the commercially available Amplite Fluorimetric Assay following treatment with avocatin B (10 mmol/L), palmitate (175 mmol/L), or avocatin B and palmitate according to the manufacturers protocol. Data, percentage of NADH compared with vehicle control-treated cells±SD; (H) GSH was measured in TEX cells in the presence or absence of NAC using a commercially available fluorimetric assay following treatment with avocatin B (10 mmol/L), according to the manufacturers protocol. Data, percentage of GSH compared with vehicle control-treated cells±SD; (I) TEX cells were treated with 10 mmol/L avocatin B in the presence or absence of NAC and colonies were counted as described in Example 1. All experiments were performed three times in triplicate, and representative figures are shown. *p<0.05; **p<0.01; ***p<0.001; (J) Avocatin B's activity in the OCI-AML2 cells (top panel NADH; middle panel (NADPH) and bottom (ROS).

FIG. 7: Avocatin B increases ROS. TEX cells were treated with 10 μM avocatin B and DCFH-DA and DHE were measured at increasing time points by flow cytometry. Raw data showing the homogenous cell shift in DCFH-DA or DHE are shown.

FIG. 8: Jurkat T cells cultured in ethidium bromide medium have reduced mitochondria with decreased function. (A) Jurkat T cells were cultured in ethidium bromide media for 60 days and mitochondria were detected by nonyl acridine orange (NAO) staining, which binds to the mitochondria specific lipid, cardiolipin. (Left panel) Jurkat T cells demonstrate positive NAO staining whereas (right panel) Jurkat-EtBr cells demonstrate a drastic reduction in NAO staining (˜87%); and (B) an absence of mitochondrial respiration.

FIG. 9: Mitochondria are functionally important for avocatin B-induced death. (A) Jurkat T cells were cultured in 50 ng/ml of ethidium bromide, 100 mg/ml sodium pyruvate and 50 μg/ml uridine for 60 days to create Jurkat-EtBr cells which lack functional mitochondrial. To confirm that Jurkat-EtBr cells lack mitochondria, the mitochondria specific markers adenine nucleotide translocator (ANT) and complex I (ND1) were measured by Western blotting. (B) Avocatin B's activity was tested in cells with (JURK) and with reduced (JURK-EtBr) mitochondria. Viability was measured by the ANN/PI assay and flow cytometry and data are presented as mean percentage of live cells (i.e., ANN/PI⁻)±SD from representative experiments. (C) TEX cells were grown in normoxic (21% O₂) or hypoxic (1% O₂) conditions and treated with avocatin B (2 μM), antimycin A (1 μM), rotenone (3 μM), daunorubicin (5 nM) or cytarabine (4 nM). Cell viability was measured by the sulforhodamine B assay, as described in the methods, following a 72 hour incubation period. Data are presented as percent viable. (D) Uncoupling protein 2 (UCP2) was measured in whole cell lysates of avocatin B (10 μM) or DMSO-control treated TEX cells by Western blotting. Arbitrary units (AU) are presented as fold change (compared to the 6 hour time point) and were calculated by dividing each treatment lane by the densitometry of its loading control. Densitometry was calculated as outlined in the methods. All experiments were performed three times in triplicate, representative figures are shown. *p<0.05; **p<0.01***; p<0.001.

FIG. 10: Avocatin B's cytotoxicity is dependent on [O₂]. TEX cells were grown in normoxic (21% O₂) or hypoxic (3% and 1% O₂) conditions and incubated with avocatin B (2 μM) for 72 hours. Cell viability was measured by the sulforhodamine B assay, as described in the methods. Data are presented as mean percentage of live cells±SD. All experiments were performed three times in triplicate. ***; p<0.001.

FIG. 11: CPT1 is functionally important for avocatin B induced death. (A) TEX cells were incubated with increasing concentrations of the CPT1 inhibitor etomoxir for 72 hours. (B) Avocatin B's (10 μM) activity was tested in the presence of etomoxir (100 μM; which does not impart toxicity) or (C) (bottompanel) in cells lacking CPT1. (top panel) mRNA expression demonstrating knockdown of CPT1. Unless otherwise noted, viability was measured by the ANN/PI assay and flow cytometry and data are presented as mean percentage of live cells (i.e., ANN/PI⁻)±SD from representative experiments. All experiments were performed three times in triplicate, representative figures are shown. *p<0.05; **p<0.01***; p<0.001. (D) Avocatin B's (10 mmol/L) activity was tested using colony assays in the presence of etomoxir (100 mmol/L; which does not impart toxicity); (E). NADPH was tested in CPT1 knockout cells following avocatin B (10 mmol/L) treatment. Data, percentage of NADPH relative to control. Unless otherwise noted, viability was measured by the Annexin V/PI assay and flow cytometry; data, mean percentage of live cells (i.e., Annexin \t/PI⁻)±SD from representative experiments. All experiments were performed three times in triplicate, and representative figures are shown.***, P<0.001.

FIG. 12: Avocatin A, Avoadyne and Avocadyne Acetate induce ROS production similar to Avocatin B.

### DETAILED DESCRIPTION OF THE DISCLOSURE

### I. Definitions

The term “compounds of Formula (I) and/or (II)” as used herein means compounds selected from compounds having the structure:

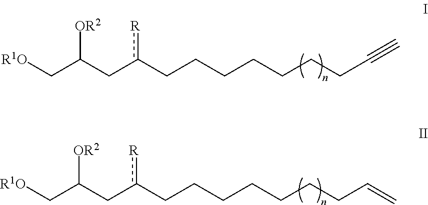

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers, or solvates thereof, as well as mixtures thereof.

The term “compounds of Formula (I)” as used herein means compounds selected from compounds having the structure:

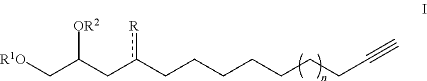

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers, or solvates thereof, as well as mixtures thereof.

The term “compounds of Formula (II)” as used herein means compounds having the structure:

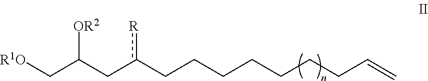

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers, or solvates thereof, as well as mixtures thereof.

As used herein, the term “compounds of Formula (I) and/or (II)” and/or isomers, stereoisomers, or solvates thereof, as well as mixtures thereof is defined to include all forms of the “compounds of Formula (I) and/or (II)”, including isomers, stereoisomers, or solvates thereof, and any pharmaceutically acceptable salts, crystalline and non-crystalline forms, polymorphs metabolites, as well as mixtures thereof. Similarly, the term “compounds of Formula (I)” is defined to include all forms of the “compounds of Formula (I)”, including isomers, stereoisomers, or solvates thereof and any pharmaceutically acceptable salts, crystalline and non-crystalline forms, polymorphs, metabolites, as well as mixtures thereof and the term “compounds of Formula (II)” is defined to include all forms of the “compounds of Formula (II)”, including isomers, stereoisomers, or solvates thereof, and any pharmaceutically acceptable salts, crystalline and non-crystalline forms, polymorphs, metabolites, as well as mixtures thereof. Further, for individual compounds and compositions disclosed herein, for example avocadyne and avocatin B respectively, the terms “avocadyne” and “avocatin B” each include isomers, stereoisomers, and solvates thereof and any crystalline and non-crystalline forms, polymorphs, metabolites, as well as mixtures thereof.

The term “avocatin B” as used herein means a mixture of: a compound of Formula (I) having the structure:

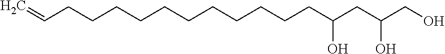 [SMILES: C=CCCCCCCCCCCCC(O)CC(O)CO]

and/or an isomer, stereoisomer, or solvate thereof, and

a compound of Formula (II) having the structure:

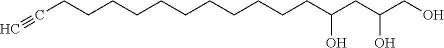 [SMILES: C#CCCCCCCCCCCCC(O)CC(O)CO]

and/or an isomer, stereoisomer, or solvate thereof. Avocatin B is an avocado-derived lipid mixture of two 17-carbon avocatin lipids, namely avocadene and avocadyne. The mixture can comprise for example a 1:1 ratio of the avocadene and avocadyne compounds. In Alves et al.²⁰, an extract comprising Avocatin B contained about 69% of the avocadene compound and 17% of the avocadyne compound.

The term “avocatin A” as used herein means a mixture of a compound of Formula (I) having the structure:

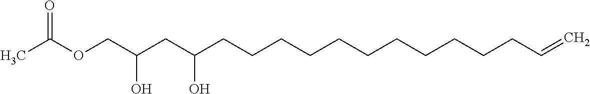 [SMILES: C=CCCCCCCCCCCCC(O)CC(O)COC(C)=O]

and/or an isomer, stereoisomer, or solvate thereof, and a compound of Formula (II) having the structure:

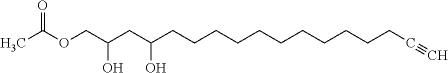 [SMILES: C#CCCCCCCCCCCCC(O)CC(O)COC(C)=O]

and/or an isomer, stereoisomer, or solvate thereof. Avocatin A is an avocado-derived lipid mixture of two 17-carbon avocatin lipids, namely avocadene acetate and avocadyne acetate. The mixture can comprise for example a 1:1 ratio of the avocadene acetate and avocadyne acetate compounds. In Alves et al.²⁰, the Avocatin A composition was identified as a mixture comprising 44% of the avocadene acetate compound and 50% of the avocadyne acetate compound.

The term “avocadene” as used herein means a compound having the structure:

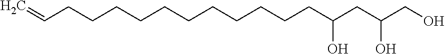 [SMILES: C=CCCCCCCCCCCCC(O)CC(O)CO]

and/or an isomer, stereoisomer, or solvate thereof.

The term “avocadene acetate” as used herein means a compound having the structure:

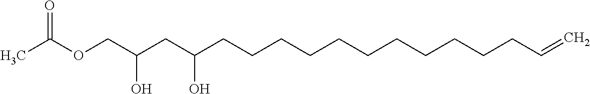 [SMILES: C=CCCCCCCCCCCCC(O)CC(O)COC(C)=O]

and/or an isomer, stereoisomer, or solvate thereof.

The term “avocadenone acetate” as used herein means a compound having the structure:

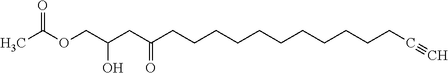 [SMILES: C#CCCCCCCCCCCCC(=O)CC(O)COC(C)=O]

and/or an isomer, stereoisomer, or solvate thereof.

The term “avocadyne” as used herein means a compound having the structure:

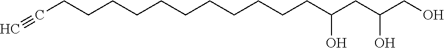 [SMILES: C#CCCCCCCCCCCCC(O)CC(O)CO]

and/or an isomer, stereoisomer, or solvate thereof.

The term “avocadyne acetate” as used herein means a compound having the structure:

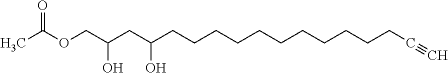 [SMILES: C#CCCCCCCCCCCCC(O)CC(O)COC(C)=O]

and/or an isomer, stereoisomer, or solvate thereof.

The term “avocadynone acetate” as used herein means a compound having the structure:

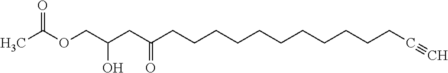 [SMILES: C#CCCCCCCCCCCCC(=O)CC(O)COC(C)=O]

and/or an isomer, stereoisomer, or solvate thereof.

The term “chemotherapeutic” as used herein refers to cytotoxic drug agent that is used for example for the treatment of leukemia and which does not accumulate in the mitochondria. It can include for example approved leukemia chemotherapeutics and experimental chemotherapeutics, for example but not limited to those in clinical trials.

The term “cytarabine” as used herein includes a compound having the structure:

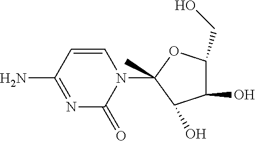 [SMILES: C[C@@]1(n2ccc(N)nc2=O)O[C@H](CO)[C@@H](O)[C@@H]1O]

and/or a pharmaceutically acceptable salt, solvate or prodrug thereof, as well as mixtures thereof.

The term “daunorubicin” as used herein includes a compound having the structure:

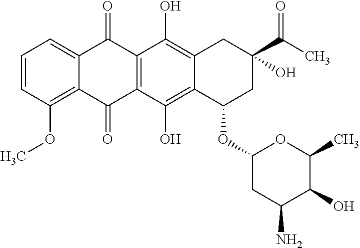 [SMILES: COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1]

and/or a pharmaceutically acceptable salt, solvate or prodrug thereof, as well as mixtures thereof.

The term “doxorubicin” as used herein includes a compound having the structure:

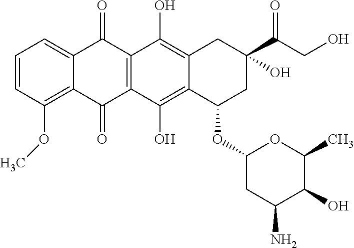 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

and/or a pharmaceutically acceptable salt, solvate or prodrug thereof, as well as mixtures thereof.

The term “leukemia” as used herein means any disease involving the progressive proliferation of abnormal leukocytes found in hematopoietic tissues, other organs and usually in the blood in increased numbers. Leukemia includes, but is not limited to, acute myeloid leukemia (AML), acute lymphocytic leukemia (ALL), chronic lymphocytic leukemia (CLL) and chronic myelogenous leukemia (CML).

The term “leukemia stem cell” as used herein refers to leukemia progenitor cells, leukemia initiating cells and/or leukemia stem cells also referred to for example as “leukemia primitive cell”.

The term “pharmaceutically acceptable salt” means an acid addition salt or a basic addition salt which is suitable for, or compatible with, the treatment of patients.

The term “pharmaceutically acceptable acid addition salt” as used herein means any non-toxic organic or inorganic acid salt of any basic compound. Basic compounds that form an acid addition salt include, for example, compounds comprising an amine group. Illustrative inorganic acids which form suitable salts include hydrochloric, hydrobromic, sulfuric and phosphoric acids, as well as metal salts such as sodium monohydrogen, orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

Where the compounds according to the disclosure possess one or more than one asymmetric centres, they may exist as “stereoisomers”, such as enantiomers and diastereomers. It is to be understood that all such stereoisomers and mixtures thereof in any proportion are encompassed within the scope of the present disclosure. It is to be understood that, while the stereochemistry of the compounds of the disclosure may be as provided for in any given compound shown herein, such compounds may also contain certain amounts (e.g. less than 20%, less than 10%, less than 5%) of compounds having alternate stereochemistry.

The term “protecting group” and “protective group” as used herein, are interchangeable and refer to an agent used to temporarily block one or more desired functional groups in a compound with multiple reactive sites. In certain embodiments, a protecting group has one or more, or all of the following characteristics: a) is added selectively to a functional group in good yield to give a protected substrate; b) is stable to reactions occurring at one or more of the other reactive sites; and c) is selectively removable in good yield by reagents that do not attack the regenerated, deprotected functional group.

As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “hydroxyl protecting group”, as used herein, refers to an agent used to temporarily block one or more desired hydroxyl reactive sites in a multifunctional compound. In an aspect, hydroxyl protecting groups also possess the characteristics exemplified for a protecting group above, and certain exemplary hydroxyl protecting groups are also detailed in Chapter 2 in Greene, T. W., Wuts, P. G in “Protective Groups in Organic Synthesis”, Third Edition, John Wiley & Sons, New York: 1999, the entire contents of which are hereby incorporated by reference.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

The term “subject” as used herein includes all members of the animal kingdom including mammals, and suitably refers to humans.

The term “inducing cytotoxicity in a cell” as used herein means causing cell damage that results in cell death.

The term “cell death” as used herein includes all forms of cell death including necrosis and apoptosis.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage leukemia can be treated to prevent progression or metastases, or alternatively a subject in remission can be treated with a compound or composition described herein to prevent recurrence.

As used herein, to “inhibit” or “suppress” or “reduce” a function or activity, such as for example mitochondrial fatty acid oxidation activity, is to reduce the function or activity when compared to a control, an otherwise same conditions except for a condition or parameter of interest, or alternatively, as compared to another condition. The terms “inhibitor” and “inhibition”, in the context of the present application, are intended to have a broad meaning and encompass compounds of Formula (I) which directly or indirectly (e.g., via reactive intermediates, metabolites and the like) act on for example the mitochondrial fatty acid oxidation pathway.

The term “synergistic” as used herein means the enhanced or magnified effect of a combination on at least one property compared to the additive individual effects of each component of the combination. For example, compounds that induce cell death by the same mechanism would not be expected to have more than additive effect. Synergism can be assessed and quantified for example by analyzing the Data by the Calcusyn median effect model where the combination index (CI) indicates synergism (CI<0.9), additively (CI=0.9-1.1) or antagonism (CI>1.1). Cis of <0.3, 0.3-0.7, 0.7-0.85, 0.85-0.90, 0.90-1.10 or >1.10 indicate strong synergism, synergism, moderate synergism, slight synergism, additive effect or antagonism, respectively. The CI is the statistical measure of synergy.

As used herein, the term “dosage form” refers to the physical form of a compound or composition for example comprising a compound and/or mixture of compounds of the disclosure, and includes without limitation liquid and solid dosage forms including, for example tablets, including enteric coated tablets, caplets, gelcaps, capsules, ingestible tablets, buccal tablets, troches, elixirs, suspensions, syrups, wafers, resuspendable powders, liquids, solutions as well as injectable dosage forms, including, for example, sterile solutions and sterile powders for reconstitution, and the like, that are suitably formulated for injection.

The term “dosage” as used herein means an amount or quantity of a compound or composition contacted with a cell, administered to a subject or for administration to a subject.

As used herein, the term “effective amount” or “therapeutically effective amount” means an amount effective, at dosages and for periods of time necessary to achieve the desired result. For example in the context or treating leukemia, an effective amount (e.g. of a compound of Formula (I) and/or (II) and optionally a chemotherapeutic) is an amount that, for example, induces remission, reduces leukemia burden, and/or prevents leukemia spread compared to the response obtained without administration of the compound. Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like. The phrase “therapeutically effective” is intended to qualify the amount of active ingredients used in the treatment of a disease or disorder. This amount will achieve the goal of reducing or eliminating the said disease or disorder.

The term “therapeutically acceptable” refers to those compounds (or salts, prodrugs, solvates, etc.) which are suitable for use in contact with the tissues of patients without undue toxicity, irritation, and allergic response, are commensurate with a reasonable benefit/risk ratio, and are effective for their intended use.

The term “administered” as used herein means administration of a therapeutically effective dose of a compound or composition to a subject such as a mammal, preferably a human or described herein to a cell for example a cell either in cell culture or in a patient. As used herein, “contemporaneous administration” and “administered contemporaneously” means that two substances (or more than two substances) are administered to a subject such that they are both biologically active in the subject at the same time. The exact details of the administration will depend on the pharmacokinetics of the two substances in the presence of each other, and can include administering one substance within 24 hours of administration of the other, if the pharmacokinetics are suitable or during the course of a treatment regimen. In particular embodiments, two substances will be administered substantially simultaneously, i.e. within minutes of each other, or in a single composition that comprises both substances.

As used herein a “combination” means two or more therapeutic agents to treat a therapeutic condition or disorder described in the present disclosure, optionally in a single composition present for example in a fixed ratio of active ingredients or in separate dosage forms for each active ingredient.

The term “mitochondrial mass” as used herein refers to the overall number and/or weight of mitochondria in a cell or number of cells. Mitochondrial mass may be determined or characterized, for example, by incubating cells with Mitotracker Green FM dye, subsequently performing flow cytometry, and determining the median fluorescence intensity of the cells. Mitochondrial mass may also be determined or characterized by incubating cells with Mitotracker Green FM dye, subsequently performing confocal scanning laser microscopy, and quantifying the fluorescence levels using an image software, for example ImageJ (see for example Agnello et al. A method for measuring mitochondrial mass and activity. Cytotechnology Vol 56(3):145-149). The mitochondrial mass of a cell or average mitochondrial mass of a number of cells, for example, in a sample taken from a subject with leukemia, can be compared to a mitochondrial mass of a control cell or number of cells in a sample taken for example from a control subject.

The term “decrease or inhibit mitochondrial fatty acid oxidation” as used herein means to reduce compared to an untreated cell, reflected for example by a reduction of NAPDH, NADH or GSH levels and an increase in reactive oxygen species (ROS) levels.

The term “nicotinamide adenine dinucleotide phosphate” or “NADPH” as used herein refers to a cofactor involved in various biochemical pathways such as catabolic processes during cell proliferation and is an important mitochondrial antioxidant. Fatty acid oxidation produces acetyl-CoA which enters the tricarboxylic acid cycle to produce NADPH. Inhibition of fatty acid oxidation can therefore lead to decreased NADPH production and reduced antioxidant capacity.

The term “nicotinamide adenine dinucleotide hydrogen” or “NADH” as used herein refers to a dinucleotide involved in various biochemical pathways and energy production. Fatty acid oxidation produces acetyl-CoA which enters the TCA cycle to produce NADH, which fuels oxidative phosphorylation, and NADPH. It can exist in two forms NAD+ and NADH.

The term “control” as used herein refers to a suitable comparator subject, sample, cell or cells such as a non-cancerous subject, or a blood sample, cell or cells from such a subject, for comparison to a cancer subject, sample (e.g. test sample) cell or cells from a cancer subject; or an untreated subject, cell or cells, for comparison to a treated subject, cell or cells, according to the context. For example, a control for comparing mitochondrial mass includes for example non-cancerous cells such as normal CD34+ bone marrow-derived hematopoietic cells, for example in a blood sample taken from a control subject free of leukemia and/or leukemia cells known to have low and/or about normal mitochondrial mass. Control can also refer to a value representative of a control subject, cell and/or cells and/or a population of subjects, for example representative of a normal mitochondrial mass.

The term “sample” as used herein refers to any biological fluid comprising a cell, a cell or tissue sample from a subject including a sample from a test subject, i.e. a test sample, such as from a subject whose mitochondrial mass is being tested, for example, a subject with leukemia, wherein the test sample comprises leukemia cells, and a control sample from a control subject, e.g., a subject without leukemia, whose mitochondrial mass is being tested. For example, the sample can comprise a blood sample, for example a peripheral blood sample, a fractionated blood sample, a bone marrow sample, a biopsy, a frozen tissue sample, a fresh tissue specimen, a cell sample, and/or a paraffin embedded section. As an example, wherein the cancer is AML, the sample comprises mononuclear cells.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed ended terms that specify the presence of stated features, elements, components, groups, integers, and/or steps, and also exclude the presence of other unstated features, elements, components, groups, integers and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

More specifically, the term “about” means plus or minus 0.1 to 50%, 5-50%, or 10-40%, 10-20%, 10%-15%, preferably 5-10%, most preferably about 5% of the number to which reference is being made.

As used in this specification and the appended claims, the singular forms “a”, “an” and “the” include plural references unless the content clearly dictates otherwise. Thus for example, a composition containing “a compound” includes a mixture of two or more compounds. It should also be noted that the term “or” is generally employed in its sense including “and/or” unless the content clearly dictates otherwise.

In compositions comprising an “additional” or “second” component, the second component as used herein is chemically different from the other components or first component. A “third” component is different from the other, first, and second components, and further enumerated or “additional” components are similarly different.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

Further, the definitions and embodiments described are intended to be applicable to other embodiments herein described for which they are suitable as would be understood by a person skilled in the art. For example, in the above passages, different aspects of the invention are defined in more detail. Each aspect so defined can be combined with any other aspect or aspects unless clearly indicated to the contrary. In particular, any feature indicated as being preferred or advantageous can be combined with any other feature or features indicated as being preferred or advantageous.

### II. Methods, Compounds and Uses

It is disclosed herein that specific avocatin compounds, identified using a screen of a natural health product library, can induce selective toxicity toward leukemia cells.

It is further disclosed herein that compounds of Formula (I) and/or (II) can synergize with a chemotherapeutic for treating leukemia in subjects in need thereof.

Accordingly, a first aspect of the disclosure is a method of treating leukemia comprising administering to a subject in need thereof a therapeutically effective amount of a compound of Formula (I) and/or (II) having the structure:

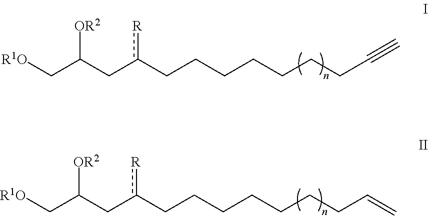

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers or solvates thereof as well as mixtures thereof.

Another aspect of the application is a use of a compound of Formula (I) and/or

(II) and/or isomers, stereoisomers or solvates thereof and/or mixtures thereof as defined herein for treating a leukemia in a subject in need thereof.

Yet another aspect of the application is a use of a compound of Formula (I) and/or (II) and/or isomers, stereoisomers or solvates thereof and/or mixtures thereof as defined herein in the preparation of a medicament for treatment of leukemia.

A further aspect of the present disclosure is a compound of Formula (I) and/or (II) and/or isomers, stereoisomers or solvates thereof and/or mixtures thereof for use in treating a leukemia in a subject in need thereof.

In an embodiment, the compound of Formula (I) and/or (II) and/or isomers, stereoisomers or solvates thereof or mixtures thereof inhibits mitochondrial fatty acid oxidation in a leukemia cell.

An embodiment includes a method of treating leukemia comprising administering to a subject in need thereof a therapeutically effective amount of a compound of Formula (I) having the structure:

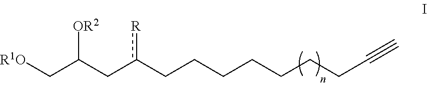

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers or solvates thereof, as well as mixtures thereof.

Another aspect of the application is a use of a compound of Formula (I) and/or isomers, stereoisomers or solvates thereof and/or mixtures thereof as defined herein for treating a leukemia in a subject in need thereof.

Yet another aspect of the application is a use of a compound of Formula (I) and/or isomers, stereoisomers or solvates thereof and/or mixtures thereof as defined herein in the preparation of a medicament for treatment of leukemia.

A further aspect of the present disclosure is a compound of Formula (I) and/or isomers, stereoisomers or solvates thereof for use in treating a leukemia in a subject in need thereof.

Another embodiment includes a method of treating leukemia comprising administering to a subject in need thereof a therapeutically effective amount of a compound of Formula (II) having the structure

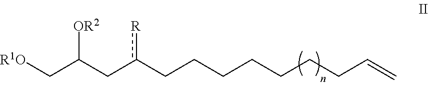

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/isomers, stereoisomers or solvates thereof, as well as mixtures thereof.

Another aspect of the application is a use of a compound of Formula (II) and/or isomers, stereoisomers or solvates thereof as defined herein for treating a leukemia in a subject in need thereof.

Yet another aspect of the application is a use of a compound of Formula (II) and/or isomers, stereoisomers or solvates thereof as defined herein in the preparation of a medicament for treatment of leukemia.

A further aspect of the present disclosure is a compound of Formula (II) and/or isomers, stereoisomers or solvates thereof for use in treating a leukemia in a subject in need thereof.

In an embodiment, the compound of Formula (I) and/or isomers, stereoisomers or solvates thereof or a mixture thereof is a compound or mixture that inhibits mitochondrial fatty acid oxidation in a leukemia cell.

In an embodiment, the compound of Formula (II) and/or isomers, stereoisomers or solvates thereof or a mixture thereof is a compound or mixture that inhibits mitochondrial fatty acid oxidation in a leukemia cell.

The compound of Formula (I) and/or (II) and/or isomers, stereoisomers or solvates thereof comprises a 13-, 15-, 17- or a 19-carbon backbone. It has been previously shown that lipids such as lipids of 16-20 carbon length can be transported into mitochondria via the membrane protein carnitine palmitoyltransferase 1 (CPT1)³⁷.

In one embodiment, the compound of Formula (I) and/or (II) and/or an isomer, stereoisomer or solvate thereof, and/or mixture thereof has n=1. In another embodiment, n=3. In another embodiment, n=5. In yet another embodiment, n=7.

In one embodiment, n=5 and the compound of Formula (I) and/or Formula (II) and/or isomers, stereoisomers or solvates thereof and/or mixtures thereof comprises a 17-carbon backbone such as avocadyne and/or avocadene. In an embodiment, the compound is a mixture comprising avocadyne, optionally avocatin B. In another embodiment, the compound is avocadyne acetate. In a further embodiment, the compound is a mixture comprising avocadyne acetate such as avocatin A.

In another embodiment, n=7 and the compound of Formula (I) and/or Formula (II) and/or or isomers, stereoisomers or solvates thereof and/or mixtures thereof comprises a 19-carbon backbone. Compounds of Formula (I) and/or Formula (II) comprising a 19-carbon backbone such as 1-nonadecene can be purchased from Santa Cruz Biotechnology, Inc. (Santa Cruz, Calif. 95060 U.S.A.) or Tokyo Chemical Industry Co., Ltd. (Tokyo, Japan).

Methods of isolating and extracting the compounds of Formula (I) and/or Formula (II) described herein from unsaponifiable matter of avocado are described in U.S. Pat. No. 6,582,688, and from fruit or vegetable source in U.S. Ser. No. 13/062,758, both of which are herein incorporated by reference in their entirety. Compounds of Formula (I) and/or (II) such as avocatin A, avocatin B, avocadyne and avocadyne acetate can be purchased from MicroSource Discovery Systems Inc. (Gaylordsville, Conn. 06755 U.S.A.) or isolated as described in Alves et al.

The compound of Formula (I) and/or (II) and/or or isomers, stereoisomers or solvates thereof can be, for example, but not limited to avocadene, avocadene acetate, avocadenone acetate, avocadyne, avocadyne acetate or avocadynone acetate and/or mixtures thereof.

In another embodiment, the compound comprises a mixture of compounds of Formula (I) and/or (II) and/or or isomers, stereoisomers or solvates thereof. For example, avocatin B is a composition comprising a mixture of avocadene and avocadyne. For example, avocatin A is a composition comprising mixture of avocadene acetate and avocadyne acetate.

In an embodiment R¹ and/or R² are optionally a protecting group.

In another embodiment, R1 is hydrogen. In an embodiment R2 is hydrogen. In an embodiment R1 is acetyl. In another embodiment, R2 is acetyl.

In yet another embodiment, R1 and R2 are hydrogen. In an embodiment, R1 is acetyl and R2 is hydrogen.

As mentioned, it was also found that Avocatin B could synergize with chemotherapeutics cytarabine and daunorubicin.

Accordingly, another aspect of the disclosure is a combination comprising a compound of Formula (I) and/or (II) and/or or isomers, stereoisomers or solvates thereof as defined above, including each compound individually described and/or as mixtures thereof, and a chemotherapeutic.

In another aspect, the combination comprising a compound of Formula (I) and/or (II) and/or or isomers, stereoisomers or solvates thereof, including each compound individually described and/or as mixtures thereof, and a chemotherapeutic are for use in the treatment of a leukemia in a subject in need thereof.

In an embodiment, the combination comprises a therapeutically effective amount of the compound of Formula (I) and/or (II) and/or isomers, stereoisomers or solvates, including each compound individually described and/or as mixtures thereof, and a therapeutically effective amount of chemotherapeutic.

Administration of a combination comprising a compound or mixture described herein of Formula (I) and/or (II) and/or or isomers, stereoisomers or solvates thereof, and/or mixtures thereof, including a compound wherein n=1, n=3, n=5 or n=7 in combination with any of wherein R1=hydrogen or acetyl and/or in combination with any of wherein R2=hydrogen or acetyl, and a chemotherapeutic may allow for a reduction of the dose of one or more compounds of Formula (I) and (II) and/or or isomers, stereoisomers or solvates thereof and/or mixtures thereof and/or a reduction of the chemotherapeutic. Alternatively, the combination may allow for enhanced anti-leukemic effect.

In an embodiment, the combination is for use in treating a leukemia in a subject in need thereof.

In an embodiment, the combination comprises a compound described herein of Formula (I) and/or (II) and/or isomers, stereoisomers or solvates thereof, including a compound wherein n=1, n=3, n=5 or n=7 in combination of wherein R1=hydrogen or acetyl and/or in combination with any of wherein R2=hydrogen or acetyl, and a chemotherapeutic.

In an embodiment, the chemotherapeutic is a chemotherapeutic approved for the treatment of a leukemia.

In another embodiment, the chemotherapeutic is cytarabine.

In another embodiment, the chemotherapeutic is an anthracycline compound such as for example daunorubicin, doxorubicin, mitoxantrone, idarubicin and amsacrine. In an embodiment, the anthracycline is daunorubicin. In an embodiment, the anthracycline is doxorubicin. In an embodiment, the anthracycline is mitoxantrone. In an embodiment, the anthracycline is idarubicin. In an embodiment, the anthracycline is amsacrine.

In yet another embodiment, the leukemia is selected from acute myeloid leukemia (AML), acute lymphocytic leukemia (ALL), chronic lymphocytic leukemia (CLL) and chronic myelogenous leukemia (CML).

In an embodiment, the leukemia is AML. In an embodiment, the leukemia is ALL. In an embodiment, the leukemia is CLL. In a further embodiment, the leukemia is CML.

In an embodiment, the combination comprises a compound of Formula (I) and/or an isomer, stereoisomer or solvate thereof and/or mixture thereof wherein n=1 and a chemotherapeutic.

In an embodiment, the combination comprises a compound of Formula (I) n=1 and a chemotherapeutic.

In an embodiment, the combination comprises a compound of Formula (I) and/or an isomer, stereoisomer or solvate thereof and/or mixture thereof wherein n=3 and a chemotherapeutic.

In an embodiment, the combination comprises a compound of Formula (I) wherein n=3 and a chemotherapeutic.

In an embodiment, the combination comprises a compound of Formula (I), and/or an isomer, stereoisomer or solvate thereof and/or mixture thereof wherein n=5 and a chemotherapeutic.

In an embodiment, the combination comprises a compound of Formula (I) wherein n=5 and a chemotherapeutic.

In an embodiment, the combination comprises a compound of Formula (I) and/or an isomer, stereoisomer or solvate thereof and/or mixture thereof wherein n=7 and a chemotherapeutic.

In an embodiment, the combination comprises a compound of Formula (I) wherein n=7 and a chemotherapeutic.

In an embodiment, the combination comprises a compound of Formula (I) and/or an isomer, stereoisomer or solvate thereof and/or mixture thereof wherein n=7 and cytarabine.

In an embodiment, the combination comprises a compound of Formula (I) and/or an isomer, stereoisomer or solvate thereof and/or mixture thereof wherein n=7 and daunorubicin.

In an embodiment, the combination comprises a compound of Formula (I) and/or an isomer, stereoisomer or solvate thereof and/or mixture thereof wherein n=7 and doxorubicin.

In an embodiment, the combination comprises a compound of Formula (I) and/or an isomer, stereoisomer or solvate thereof and/or mixture thereof wherein n=7 and mitoxantrone. In an embodiment, the combination comprises a compound of Formula (I) and/or an isomer, stereoisomer or solvate thereof and/or mixture thereof wherein n=7 and idarubicin. In an embodiment, the combination comprises a compound of Formula (I) and/or an isomer, stereoisomer or solvate thereof and/or mixture thereof wherein n=7 and amsacrine.

In an embodiment, the combination comprises avocadyne and a chemotherapeutic. In an embodiment, the combination comprises avocadyne acetate and a chemotherapeutic. In an embodiment, the combination comprises avocadynone acetate or a mixture comprising avocadynone acetate and a chemotherapeutic.

In an embodiment, the combination comprises avocatin B and cytarabine.

In another embodiment, the combination comprises avocatin B and daunorubicin.

In another embodiment, the combination comprises avocatin B and doxorubicin.

In an embodiment, the combination comprises avocadyne and cytarabine.

In an embodiment, the combination comprises avocadyne acetate and cytarabine.

In another embodiment, the combination comprises avocadyne and daunorubicin.

In another embodiment, the combination comprises avocadyne acetate and daunorubicin.

In another embodiment, the combination comprises avocadyne and doxorubicin.

In another embodiment, the combination comprises avocadyne acetate and doxorubicin.

In yet a further embodiment, the combination comprises avocadyne and mitoxantrone. In yet a further embodiment, the combination comprises avocadyne acetate and mitoxantrone.

In yet a further embodiment, the combination comprises avocadyne and idarubicin. In yet a further embodiment, the combination comprises avocadyne acetate and idarubicin.

In yet a further embodiment, the combination comprises avocadyne and amsacrine. In yet a further embodiment, the combination comprises avocadyne acetate and amsacrine.

In an embodiment, the compound of Formula (I) and/or (II) and/or isomers, stereoisomers or solvates thereof, and/or mixtures thereof is a pharmaceutically acceptable salt thereof.

In an embodiment, the compound is a compound of Formula (I) and/or (II) and/or a mixture thereof.

In and embodiment, the compound is compound of Formula(I) and/or Formula

(II) and/or a solvate thereof.

In and embodiment, the compound is compound of Formula(I) and/or Formula (II) and/or an isomer thereof.

In and embodiment, the compound is compound of Formula(I) and/or Formula (II) and/or a stereoisomer thereof.

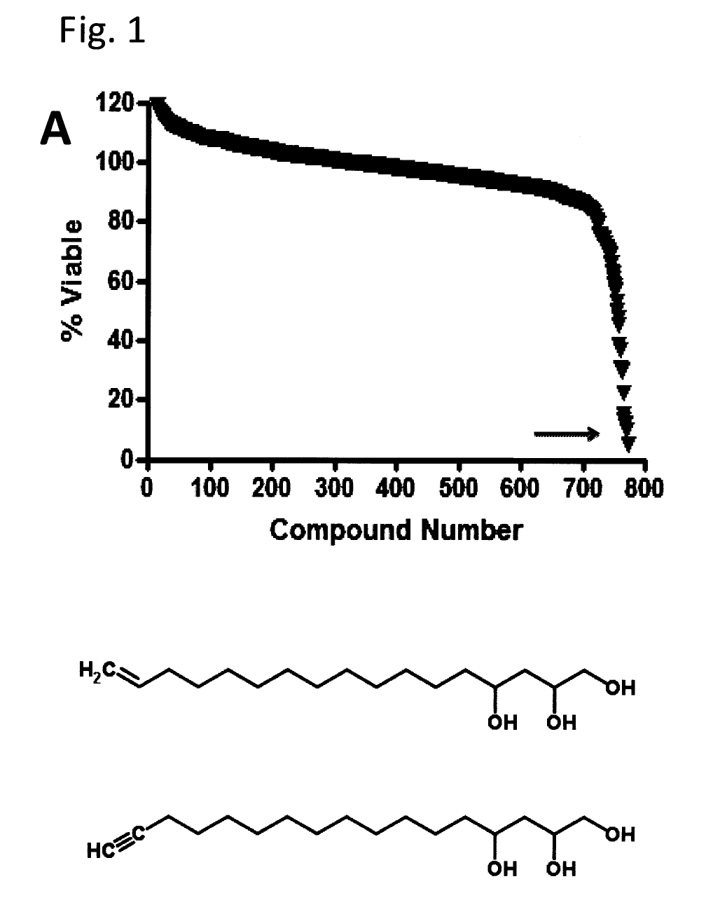

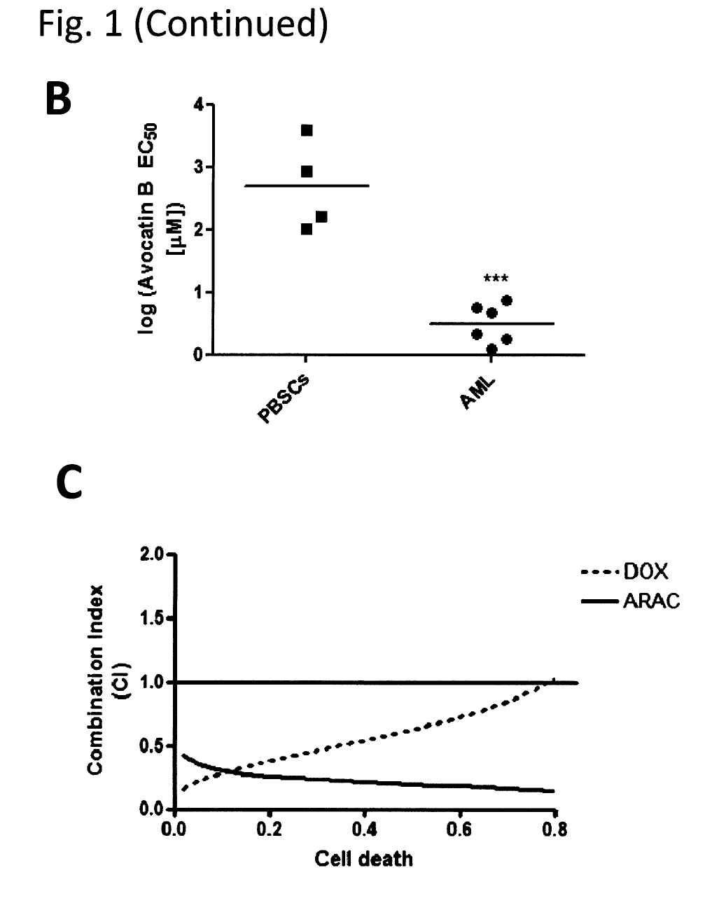

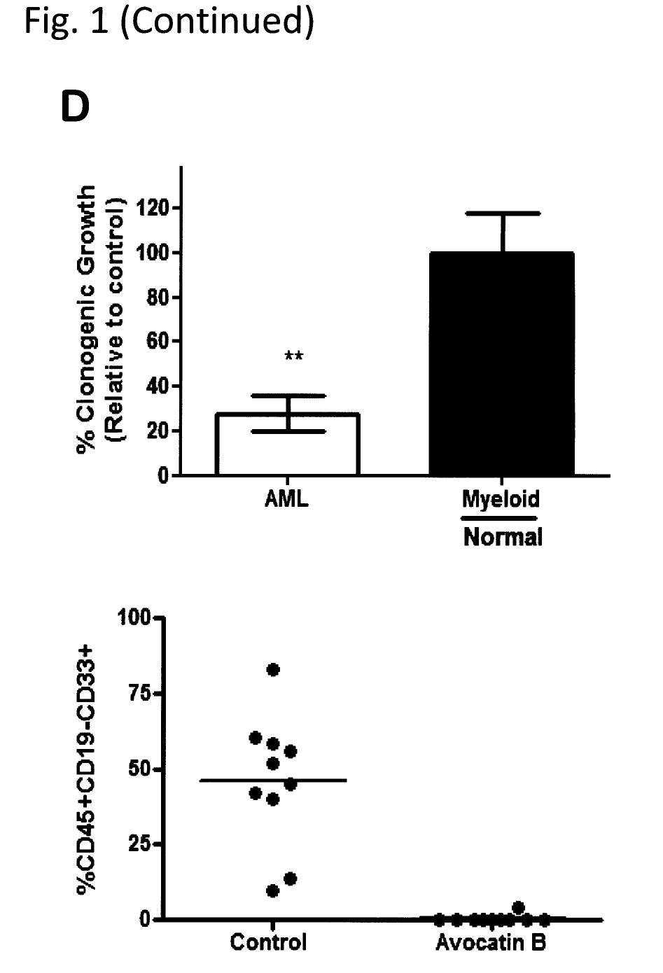

Compounds having the structure of Formula (I) and/or (II) and/or an isomer, stereoisomer or solvates thereof and/or mixtures thereof as shown herein selectively induce cytotoxicity in leukemia cells while sparing normal cells. As shown in FIG. 1B, avocatin B reduced the viability of primary AML patient cells with an EC50 of 3.9 μM and yet had no effect on the viability of normal peripheral blood stem cells from healthy donors at concentrations as high as 20 μM. Likewise, adding avocatin B to a culture medium reduced the clonogenic growth of AML patient cells but had no effect on normal cells (see FIG. 1D, top panel). Using a mouse xenotransplant model it was also demonstrated that primary AML cells treated with avocatin B exhibited a reduction in the ability to engraft in the marrow of immune deficient mice compared to non-treated cells (see FIG. 1D, bottom panel).

It is demonstrated, that avocatin B can inhibit and kill leukemic stem cells. Accordingly a further aspect is a method of inhibiting leukemia stem cells comprising contacting administering to a subject in need thereof a compound of Formula (I) and/or (II) having the structure:

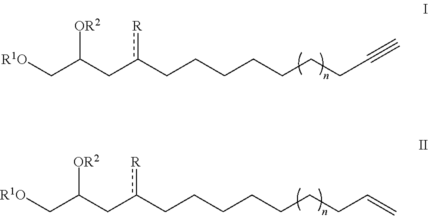

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers or solvates thereof and/or mixtures thereof.

In an embodiment, the compound is any compound of Formula (I) including individual compounds described herein and mixtures as well as combinations with a chemotherapeutic.

In an embodiment, the compound is any compound of Formula (II) including individual compounds described herein and mixtures as well as combinations with a chemotherapeutic.

Another aspect of the disclosure is a composition comprising a compound of Formula (I) and/or (II) and/or isomers, stereoisomers or solvates thereof and/or mixture thereof as defined herein, including individual compounds described herein and mixtures as well as combinations with a chemotherapeutic for use in a method, use, combination or kit described herein.

In an embodiment, the composition is an extract, optionally from avocado pear seeds, obtained for example as described in Alves et al.

In an embodiment, the composition is a pharmaceutical composition.

In an embodiment, the composition comprises a therapeutically effective amount of the compound of Formula (I) and/or (II) and/or isomers, stereoisomers or solvates thereof and/or mixture thereof thereof as defined above and one or more suitable excipients, diluents, buffers, carriers or vehicles. In an embodiment, the composition comprises a compound of Formula (I) and/or isomers, stereoisomers or solvates thereof and/or mixture thereof, including anyone of the Formula (I) compounds described individually herein. In an embodiment, the composition comprises a compound of Formula (II) and/or isomers, stereoisomers or solvates thereof and/or mixture thereof, including anyone of the Formula (II) compounds described individually herein.

In an embodiment, the excipient is a nonionic detergent. Optionally the detergent is polysorbate 20 or polysorbate 80. In an embodiment, the composition comprises cyclodextran. In an embodiment, the diluent is phosphate buffered saline.

In another embodiment, the pharmaceutical composition is for use in treating leukemia in a subject in need thereof.

In a further embodiment, the pharmaceutical composition is in a dosage form selected form a solid dosage form and a liquid dosage form.

In yet another embodiment, the pharmaceutical composition is administered by parenteral, intravenous, subcutaneous, intramuscular, intraspinal, intracisternal, intraperitoneal, or oral administration.

For example, the compounds may be formulated for parenteral administration by injection, e.g., by bolus injection or continuous infusion, either to the body or to the site of a disease. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. To administer the therapeutic compound by other than parenteral administration, it may be necessary to coat the compound with, or co-administer the compound with a material to prevent its inactivation (for example, via liposomal formulation).

Sterile injectable solutions can be prepared by incorporating the therapeutic compound in the required amount in an appropriate solvent with one or a combination of ingredients enumerated above, as required, followed by filtered sterilization. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

In an embodiment, the pharmaceutical composition comprises an oral dosage form. In another embodiment, the pharmaceutical composition comprises an injectable dosage form. In further embodiment, the dosage form is suitable for oral administration. In yet another embodiment, the dosage is suitable for injection.

Another aspect of the disclosure is a test assay for identifying putative combinations for treating leukemia comprising:
a. contacting a cell with a compound of Formula (I) and/or (II) having the structure:

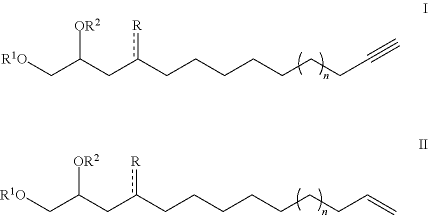

wherein:
   [symbol] represents a single or a double bond;
   R is OH when C [symbol] R is C—R, and R is O when C [symbol] R is C═R;
   n is 1, 3, 5 or 7; and
   R¹ and R² are independently hydrogen or acetyl,
and/or or isomers, stereoisomers or solvates thereof and/or mixtures thereof in the presence and in the absence of a test agent;
b. measuring the viability of the cell in the presence of the test agent and in the absence of the test agent, optionally using a MTS assay;
c. determining if the compound of Formula (I) and/or (II) and/or a or isomers, stereoisomers or solvates thereof and/or mixtures thereof in combination with the test agent exhibit synergistic cytotoxicity; and
d. optionally testing synergistic combinations is a second viability assay.

In an embodiment, the cell is contacted with a compound of Formula (I) and/or or isomers, stereoisomers or solvates thereof and/or mixtures of compounds of Formula (I). In an embodiment, the compound of Formula (I) has n=7. In a further embodiment, the compound is avocatin B. In another embodiment, the compound is avocadyne or is a mixture comprising avocadyne. In another embodiment, the compound is avocadyne acetate or is a mixture comprising avocadyne acetate. In another embodiment, the compound is avocadynone acetate or is a mixture comprising avocadynone acetate

In an embodiment, the compound is a compound or mixture described herein.

In an embodiment, the presence or absence of synergism is determined by measuring the combination index (CI) using a Calcusyn median effect model, wherein CI<1 is indicative of a synergistic effect, CI=1 is indicative of an additive effect and CI>1 is indicative of an antagonistic effect.

In an embodiment, the test agent is a chemotherapeutic for example an anthracycline compound.

In an embodiment, the leukemia cell is for example, but not limited to, an AML cell, an ALL cell, a CLL cell or a CML cell.

Yet another aspect relates to a method of identifying a subject with leukemia likely to benefit from administration of a compound of Formula (I) and/or (II) having the structure:

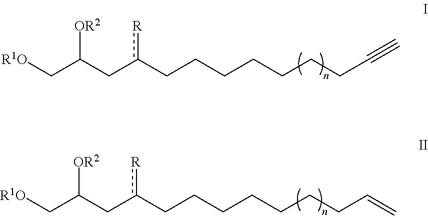

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,
and/or or isomers, stereoisomers or solvates thereof and/or mixtures thereof, and optionally a chemotherapeutic, comprising:
   a. obtaining a test sample comprising leukemia cells from the subject;
   b. determining a mitochondrial mass of the test sample; and
   c. comparing the mitochondrial mass of the test sample to a mitochondrial mass of a control;
      wherein the subject is identified as likely to benefit from administration or use of the compound of Formula (I) and/or (II) or isomers, stereoisomers or solvates thereof and/or mixtures thereof, and optionally in combination with a chemotherapeutic when the leukemia cells have an at least 2 fold increased mitochondrial mass compared to the control. In an embodiment, the subject is identified as likely to benefit by providing the subject or the subject's medical professional with a report indicating that the subject is likely to benefit from administration or use of the compound of Formula (I) and/or (II) or isomers, stereoisomers or solvates thereof and/or mixture thereof

In an embodiment, the subject is determined to likely benefit from administration of a compound of Formula (I). In an embodiment, the subject is determined to likely benefit from a compound described herein.

In an embodiment, the method further comprises administering to the subject in need thereof a therapeutically effective amount of a compound of Formula (I) and/or (II) having the structure:

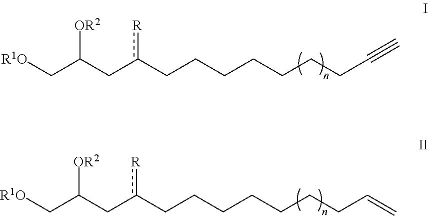

wherein:
[symbol] represents a single or a double bond;
R is OH when C [symbol] R is C—R, and R is O when C [symbol] R is C═R;
n is 1, 3, 5 or 7; and
R¹ and R² are independently hydrogen or acetyl,

and/or isomers, stereoisomers or solvates thereof and/or mixture thereof.

In an embodiment, the compound is a compound of Formula (I) and/or an isomer, stereoisomer or solvate thereof and/or mixture of compounds of Formula (I). In an embodiment, the compound of Formula (I) has n=7. In a further embodiment, the compound is avocatin B. In another embodiment, the compound is avocadyne or is a mixture comprising avocadyne. In another embodiment, the compound is avocadyne acetate or is a mixture comprising avocadyne acetate. In another embodiment, the compound is avocadynone acetate or is a mixture comprising avocadynone acetate.

In an embodiment, the compound is a compound described herein.

In an embodiment, the compounds of Formula (I) and/or (II) are administered in combination with a chemotherapeutic.

Another aspect of the disclosure is a kit comprising a compound of Formula (I) and/or (II) having the structure:

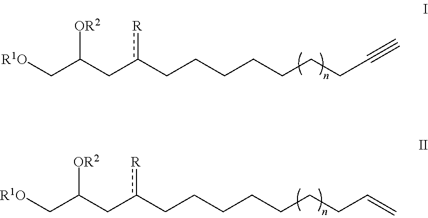

wherein:

[symbol] represents a single or a double bond;

R is OH when C [symbol] R is C—R, and R is O when C [symbol] R is C═R;

n is 1, 3, 5 or 7; and

R¹ and R² are independently hydrogen or acetyl,

and/or isomers, stereoisomers or solvates thereof and/or mixture thereof, optionally a chemotherapeutic, and/or packaging instructions for use thereof.

In an embodiment, the kit comprises a compound of Formula (I) and/or isomers, stereoisomers or solvates thereof and/or mixture of compounds of Formula (I). In an embodiment, the compound of Formula (I) has n=7. In a further embodiment, the compound is avocatin B. In another embodiment, the compound is avocadyne or is a mixture comprising avocadyne. In another embodiment, the compound is avocadyne acetate or is a mixture comprising avocadyne acetate. In a further embodiment, the compound is avocatin B. In another embodiment, the compound is avocadynone acetate or is a mixture comprising avocadynone acetate.

In an embodiment, the kit comprises compound is a compound described herein.

In an embodiment, the kit comprises instructions for carrying out a method or use described herein and comprises a, compound, composition and/or a combination described herein.

In an embodiment the kit is used for and/or comprises instructions for use for treating leukemia.

In another embodiment, the kit comprises a compound of Formula (I) or isomers, stereoisomers or solvates thereof and/or mixture thereof. In an embodiment, the compound of Formula (I) is a compound where n=5. In an embodiment, the kit comprises avocatin B, avocatin A, avocadyne, avocadyne acetate, and/or avocadynone acetate.

In another embodiment, the chemotherapeutic is cytarabine and/or an anthracycline compound. In an embodiment, the anthracycline compound is daunorubicin, doxorubicin, mitoxantrone, idarubicin or amsacrine.

It is to be understood that combinations of features described herein are contemplated and can be combined unless clearly incompatible.

The above disclosure generally describes the present application. A more complete understanding can be obtained by reference to the following specific examples.

These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

### Methods

### Cell Culture

Leukemia (OCI-AML2) cells were cultured in Iscove's Modified Dulbecco's Medium (IMDM) (Life Technologies; Grand Island, N.Y.) supplemented with 10% Fetal Bovine Serum (FBS; Seradigm; Providence, Utah) and antibiotics (100 units/ml of streptomycin and 100 μg/m of penicillin; Sigma Chemical; St. Louis, Mo.). TEX leukemia cells were cultured in 15% FBS, antibiotics and 2 mM L-glutamine (Sigma Chemical), 20 ng/ml stem cell factor and 2 ng/ml IL-3 (Peprotech; Hamburg Germany). Primary human samples (fresh and frozen) were obtained from the peripheral blood of AML patients who had at least 80% malignant cells among the mononuclear cells and cultured at 37° C. in IMDM, 20% FBS and antibiotics (see table 1a and 1 b for clinical parameters). Normal GCSF-mobilized peripheral blood mononuclear cells (PBSCs) obtained from volunteers donating peripheral blood stem cells for allotransplant were cultured similar to the primary AML samples.

### Cell Growth and Viability

Cell growth and viability was measured using the 3-(4,5-dimethylthiazol-2-yl)-5-(3-carboxymethoxyphenyl)-2-(4-sulfophenyl)-2H-tetrazolium inner salt (MTS) reduction assay (Promega; Madison, Wis.) according to the manufacturer's protocol and as previously described⁷. Cells were seeded in 96-well plates, treated with drug for 72 hours and optical density (OD) was measured at 490 nm. Cell viability was also assessed by the trypan blue exclusion assay and by Annexin V and PI staining (Biovision; Mountainview, Calif.), as previously described⁷.

### Hypoxia Experiments

Cells were transferred to hypoxic culture chambers (MACS VA500 microaerophilic workstation, H35 HypoxyWorkStation; Don Whitley Scientific; UK) in which the atmosphere consisted of residual N2, 5% H₂, 5% CO₂ and 1% or 21% O₂. Cell growth and viability were measured by the sulforhodamine B assay (Sigma Chemical) as previously described⁸ following 72 hours of incubation. Data are presented as the % change in viability from 21%-1% oxygen.

### Functional Stem Cell Assays

Clonogenic growth assays with primary AML and normal hematopoietic stem cells were performed, as previously described⁷. Briefly, CD34⁺ bone marrow-derived normal stem cells (StemCell Technologies; Vancouver, Canada) or AML mononuclear cells from patients with >80% blasts in their peripheral blood (4×10⁵ cells/ml) were treated with vehicle control or increasing concentrations of avocatin B and plated in duplicate by volume at 10⁵ cells/ml per 35 mm dish (Nunclon; Rochester, USA) in MethoCult GF H4434 medium (StemCell Technologies) containing 1% methylcellulose in IMDM, 30% FBS, 1% bovine serum albumin, 3 U/ml recombinant human erythropoietin, 10⁻⁴ M 2-mercaptoethanol (2ME), 2 mM L-glutamine, 50 ng/ml recombinant human stem cell factor, 10 ng/ml recombinant human granulocyte macrophage-colony stimulating factor and 10 ng/ml recombinant human IL-3. After 7-10 days of incubation at 37° C. with 5% CO₂ and 95% humidity, the numbers of colonies were counted on an inverted microscope with a cluster of 10 or more cells counted as one colony.

Mouse xenotransplant assays were performed as previously described^{1,9}. Briefly, AML patient cells were treated with 3.0 μM avocatin B or dimethyl sulfoxide (DMSO) (as a control) for 48 hours in vitro. Next, these cells were transplanted into femurs of sublethally irradiated, CD122 treated NOD/SCID mice and following a 6 week engraftment period, mice were sacrificed, femurs excised and bone marrow flushed and the presence of human myeloid cells (CD45⁺/CD33⁺/CD19⁻) were detected by flow cytometry.

### High-Throughput Screen

A high throughput screen of a natural product library (n=800; Microsource Discovery Systems Inc.; Gaylordsville, Conn.) was performed as previously described^{1,7}. Briefly, TEX leukemia cells (1.5×10⁴/well) were seeded in 96-well polystyrene tissue culture plates. After seeding, cells were treated with aliquots (10 μM final concentration) of the chemical library with a final DMSO concentration no greater than 0.05%. After 72 hours, cell proliferation and viability were measured by the MTS assay.

### Drug Combination Studies

The combination index (CI) was used to evaluate the interaction between avocatin B and cytarabine. TEX cells were treated with increasing concentrations of avocatin B in the presence and absence of cytarabine and after 72 hours cell viability was measured by the MTS assay. CI values, generated by the Calcusyn median effect model, were used to evaluate whether the avocatin B/cytarabine combination was synergistic, antagonistic or additive. CI values of <1 indicate synergism, CI=1 indicate additivity and CI>1 indicate antagonism^{10,11}.

Protein and mRNA Detection

Western blotting was performed as previously described¹². Briefly, whole cell lysates were prepared from treated cells, heated for 5 minutes at 95° C., and subjected to gel electrophoresis on 7.5-15% SDS-polyacrylamide gels at 150V for 85 minutes. The samples were then transferred at 25V for 45 minutes to a PVDF membrane and blocked with 5% bovine serum albumin (BSA) in Tris-buffered saline-tween (TBS-T) for 1 hour. The membrane was incubated overnight at 4° C. with the primary antibody, Poly ADP ribose polymerase (PARP)a (1:1500; Cell Signaling; Danvers, Mass.), UCP2 (1:1000; Santa Cruz Biotechnology; Dallas, Tex.), ANT (1:1000; Santa Cruz), ND1 (1:10000; Santa Cruz) or α-tubulin (loading control; 1:5000; Santa Cruz Biotechnology). Membranes were then washed and incubated with the appropriate secondary antibody (1:10000) for 1 hour at room temperature. Enhanced chemiluminescence (ECL) was used to detect proteins according to the manufacturer's instructions (GE Healthcare; Baie d'Urfe, Quebec) and luminescence was captured using the Kodak Image Station 4000MM Pro and analyzed with a Kodak Molecular Imaging Software Version 5.0.1.27. For the UCP2 blot, densitometry was determined using the imaging software and arbitrary units were calculated by dividing band intensity by its loading control (α-tubulin).

Quantitative PCR were performed as previously described⁶ in triplicate using an ABI 7900 Sequence Detection System (Applied Biosystems) with 5 ng of RNA equivalent cDNA, SYBR Green PCR Master mix (Applied Biosystems, Foster City, Calif., USA), and 400 nM of CPT1-specific primers (forward: 5′-TCGTCACCTCTTCTGCCTTT-3′ (SEQ ID NO: 1), reverse: 5′-ACACACCATAGCCGTCATCA-3′, (SEQ ID NO:2)). Relative mRNA expression was determined using the ΔΔCT method as previously described⁶.

### Assessment of Fatty Acid Oxidation and Mitochondrial Respiration

Measurement of oxygen consumption rates were performed using a Seahorse XF24 extracellular flux analyzer (Seahorse Bioscience; North Billerica, Mass.). TEX cells were cultured in α-Minimum Essential Medium (Life Technologies) containing 1% FBS and plated at 1×10⁵ cells/well in poly-L-Lysine (Sigma Chemical) coated XF24 plates. Cells were incubated with etomoxir (100 μM; Sigma Chemical) or vehicle control for 30 minutes at 37° C. in a humidified atmosphere containing 5% CO₂. Next, palmitate (175 μM; Seahorse Bioscience) or avocatin B (10 μM) was added and immediately transferred to the XF24 analyzer. Oxidation of exogenous fatty acids was determined by measuring mitochondrial respiration through sequential injection of 5 μM (final concentration) oligomycin, an ATP synthase inhibitor, (Millipore, Billerica, Mass.), 5 μM CCCP, a hydrogen ion ionophore, (Sigma Chemical), and 5 μM rotenone (Millipore)/5 μM antimycin A, which inhibit complex III activity, (Sigma Chemical). Fatty acid oxidation was determined by the change in oxygen consumption following oligomycin and CCCP treatment and prior to antimycin and rotenone treatment, according to the manufacturer's protocol and as described in Abe et al. (2013)¹³. Data were analyzed with XF software (Seahorse Bioscience).

### Reactive Oxygen Species, NADH, NADPH and GSH Detection

Reactive oxygen species (ROS) were detected using 2′,7′dichlorohydrofluorescein-diacetate (DCFH-DA; Sigma Chemical) and dihyodroethidium (DHE; Sigma Chemical). DCFH-DA is hydrolyzed by intracellular esterase to produce a non-fluorescent DCFH product. It can then be oxidized by ROS to produce a highly fluorescent DCF product¹⁴. DHE is a superoxide indicator which upon contact with superoxide anions produces the fluorescent product 2-hydroxyethidium¹⁵. Following drug treatment, TEX cells (5×10⁵) were collected and washed in PBS (Sigma-Aldrich). Cells were stained with 5 μM (final concentration) DCFH-DA or 10 μM DHE and allowed to incubate for 30 minutes in a humidified atmosphere containing 5% CO₂ at 37° C. Samples were then washed in PBS and ROS was measured by flow cytometry using the Guava EasyCyte 8HT (Millipore). Data were analyzed with GuavaSoft 2.5 software (Millipore).

Nicotinamide adenine dinucleotide phosphate (NADPH), nicotinamide adenine dinucleotide (NAD) and glutathione (GSH) were measured by commercially available fluorimetric kits (e.g. Amplite™ Fluorimetric kit (AAT Bioquest; Sunnyvale, Calif.) according to the manufacturers' protocol and as previously described¹⁶, following incubation of increasing duration with avocatin B (10 μM). For NAPDH studies, cells were also incubated with palmitate (175 μM) in the presence or absence of etomoxir. For NADH and GSH studies, cells were incubated in the presence of palmitate and N-acetylcysteine (NAC; 1 mmol/L), respectively. Data are presented as a percent NAD, NADPH or GSH compared to control treated cells±SD.

### Liquid Chromatography/Mass Spectroscopy

Avocatin B's presence in mitochondria and cytosolic fractions was detected using thin film solid-phase microextraction (TF-SPME; Professional Analytical System Technology) followed by liquid chromatography-high resolution mass spectrometry analysis (LC/MS; Thermo Exactive Orbitrap mass spectrometer; Thermo Scientific; refs. 14, 15). TEX cells were treated with avocatin B or a vehicle control for 1 hour, as performed for the Seahorse Bioanalyzer experiments (i.e., assessment of fatty acid oxidation), and cytosolic and mitochondrial fractions were then isolated, as previously described (16). Fraction purity was determined by Western blot analysis for the mitochondrial-specific protein ND1 (i.e., complex 1). Next, samples were prepared by TF-SPME and then subjected to LC/MS analysis.

### Apoptosis Determination

Caspase activation, PARP cleavage, Annexin V (ANN)/Propidum Iodide (PI), and DNA fragmentation assays were performed, as previously described¹². Release of pro-apoptotic mitochondrial proteins cytochrome c and apoptosis inducing factor (AIF) were assessed using a flow cytometry-based assay, as previously described^{17,18} and these assays are further detailed below.

### Apoptosis Measurements

Apoptotic phenotype of Annexin V⁺(ANN)/Propidium Iodide (PI)⁻ was assessed using the ANN/PI assay, as previously described¹². ANN binds to surface phosphatidylserine and PI transverses only disrupted plasma membranes to intercalate with DNA. Thus, measurements of ANN⁺/PI⁻ indicate apoptosis whereas ANN⁺/PI⁺, ANN⁻/PI⁻ indicate a dead or viable cell, respectively. Analysis of the sub G1 peak was performed by assessing cell cycle as previously described⁷. Briefly, TEX cells treated for 24 hours with 10 μM avocatin B were harvested, washed with cold PBS and re-suspended in PBS and cold absolute ethanol. Cells were then treated at 37° C. for 30 min with 100 ng/mL of DNase-free RNase A (Invitrogen; Carlsbad, Calif.), washed with cold PBS, resuspended in PBS and incubated with 50 μg/mL of propidium iodine (PI) for 15 min at room temperature in the dark. DNA content was measured by flow cytometry and analyzed with the Guava Cell Cycle software (Millipore). Caspase activation was performed using a commercially available kit (Promega) and was performed according to the manufacturer's protocol. Z-VAD-FMK (Sigma Chemical) was used as a pan-caspase inhibitor. Cleavage of poly (ADP) ribose polymerase (PARP), a DNA repair enzyme and a common downstream target of active caspase 3&7 were measured as previously described¹².

### AIF and Cytochrome c Detection

To determine avocatin B's effect on the release of pro-apoptotic mitochondrial proteins cytochrome c and AIF, a flow cytometry-based assay was used as previously described^{17,18}. Briefly, pre-treated TEX cells (2×10⁵) were collected and permeabilized in ice-cold digitonin buffer (50 μg/ml, 100 mM KCl, in PBS) for 3-5 minutes on ice (until >95% cells were permeabilized, as assessed by trypan blue staining). Permeabilized cells were fixed in 4% paraformaldehyde (in PBS) for 20 minutes at room temperature, washed 3 times in PBS, and then resuspended in blocking buffer (0.05% saponin, 3% BSA in PBS) for 1 hour at room temperature. Cells were incubated overnight at 4° C. with 1:200 cytochrome c antibody or AIF antibody (Santa Cruz Biotechnology) diluted in blocking buffer, washed three times with PBS and then incubated for 1 hour at room temperature with 1:200 Alexa Fluor-488 donkey anti-mouse IgG secondary antibody (Life Technologies) diluted in blocking buffer. Cells were washed three times in PBS and analyzed by flow cytometry using the BD FACS Calibur.

### Statistical Analysis

Unless otherwise stated, the results are presented as mean±SD. Data were analyzed using GraphPad Prism 4.0 (GraphPad Software, La Jolla, Calif.). p<0.05 was accepted as being statistically significant. Drug combination data were analyzed using Calcusyn software (Biosoft; UK)

### Results

### A High-Throughput Screen for Novel Anti-AML Compounds Identifies Avocatin B.

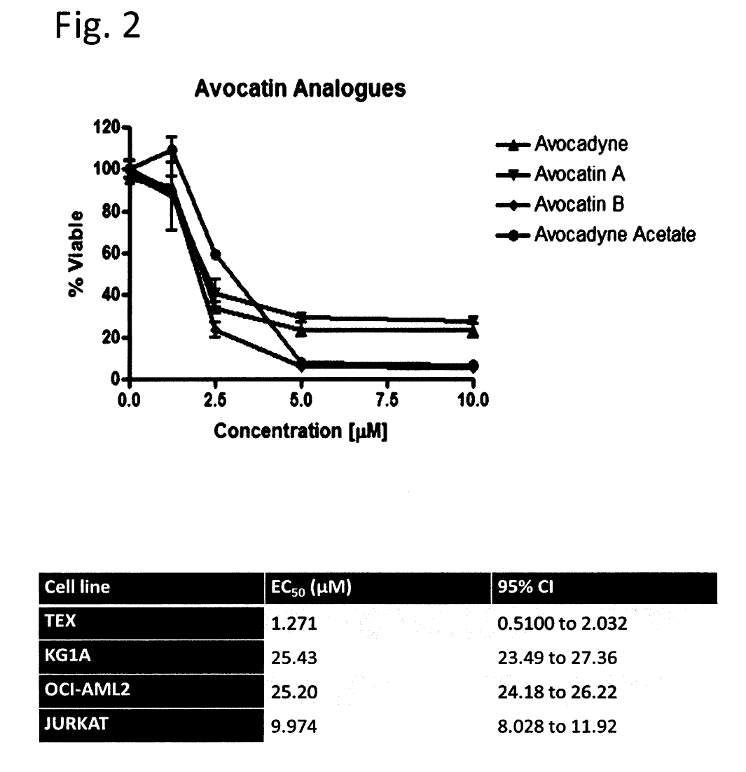

To identify novel compounds with anti-AML activity a commercially available natural health products specific library was screened against TEX leukemia cells. These cells possess several LSC properties, such as marrow repopulation and self-renewal^{1,5,6,19}. The compound which imparted the greatest reduction in viability was avocatin B (FIG. 1A; top panel, arrow indicates avocatin B). Avocatin B is a 1:1 mixture of two 17-carbon lipids derived from avocados and belongs to a family of structurally related lipids²⁰ (FIG. 1A bottom panel, Avocatin B's structure²¹). Avocatin lipid analogues were tested, and avocadyne, avocatin A, avocatin B and avocadyne acetate were found to induce cytotoxicity, and avocatin B was determined to be the most cytotoxic (EC50: 1.5±0.75 μM; FIG. 2).

Avocatin B's selectivity toward leukemia cells was validated in primary AML samples and in peripheral blood stem cells (PBSCs) isolated from GCSF-stimulated healthy donors. Avocatin B, at concentrations as high as 20 μM, had no effect on the viability of normal PBSCs (n=4). In contrast, avocatin B reduced the viability of primary AML patient cells (n=6) with an EC50 of 3.9±2.5 μM, which is similar to other recently identified compounds with anti-AML activity^{1,8,9,22} (FIG. 1B; see table 1a and 1b for patient sample characteristics).

Avocatin B was also tested in combination with cytarabine; the primary backbone of current clinical AML therapy. The Calcusyn median effect model was used to evaluate whether the avocatin B/cytarabine combination was synergistic, antagonistic or additive^{10,11}. The cytarabine-avocatin B combination synergistically induced cell death in TEX cells with combination index values of 0.20, 0.19, and 0.15, at EC 30, 50 and 80, respectively (FIG. 1C). Avocatin B was further tested in combination with an anthracycline, doxorubicin. The Calcusyn median effect model showed a synergistic effect with the doxorubicin-avocatin B combination in TEX cells. (FIG. 1C).

### Avocatin B is Selectively Toxic Toward Leukemia Progenitor and Stem Cells

Given the selectivity toward AML patient samples over normal hematopoietic cells, avocatin B's effects on functionally defined subsets of primitive human AML and normal cell populations was next assessed. Adding avocatin B (3 μM) into the culture medium reduced clonogenic growth of AML patient cells (n=3; table 1 for patient characteristics). In contrast, there was no effect on normal cells (n=3; FIG. 1D; top panel). In addition, treatment of primary AML cells with avocatin B (3.0 μM) reduced their ability to engraft in the marrow of immune deficient mice (FIG. 1D; bottom panel). Taken together, avocatin B selectively targets primitive leukemia cells (e.g. leukemia progenitor and stem cells).

### Avocatin B Induces Mitochondria-Mediated Apoptosis

The mode of avocatin B induced leukemia cell death was assessed.

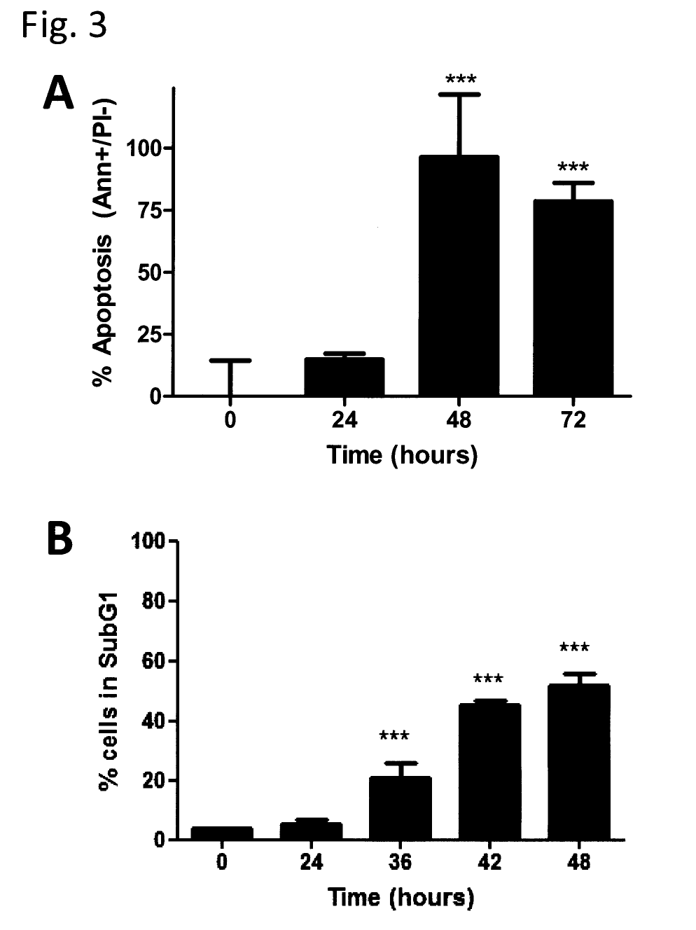

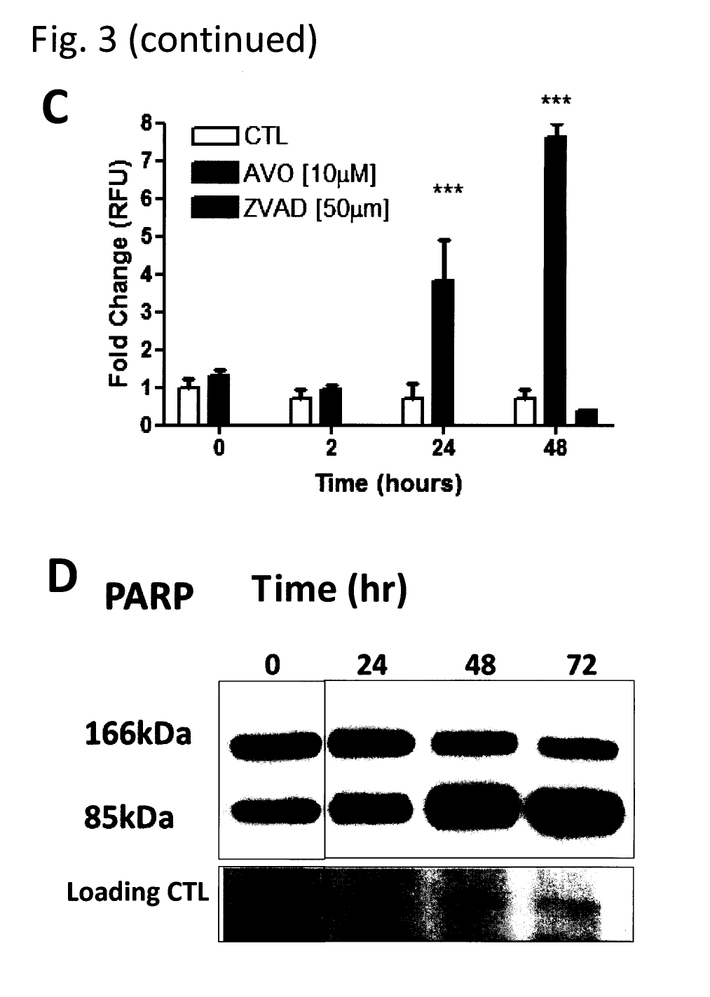

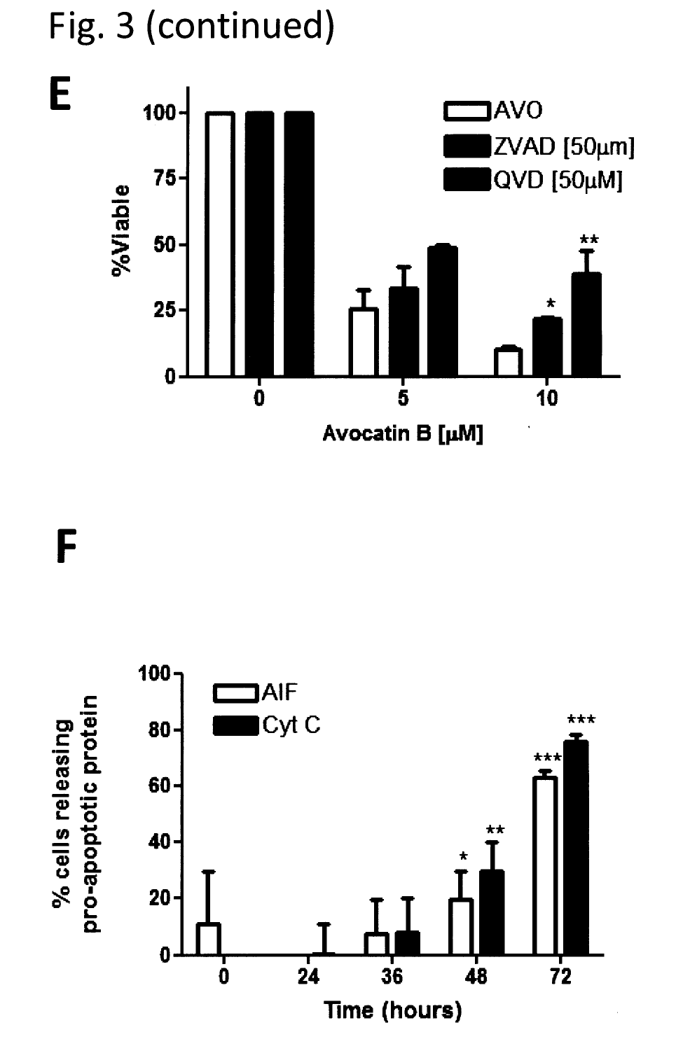

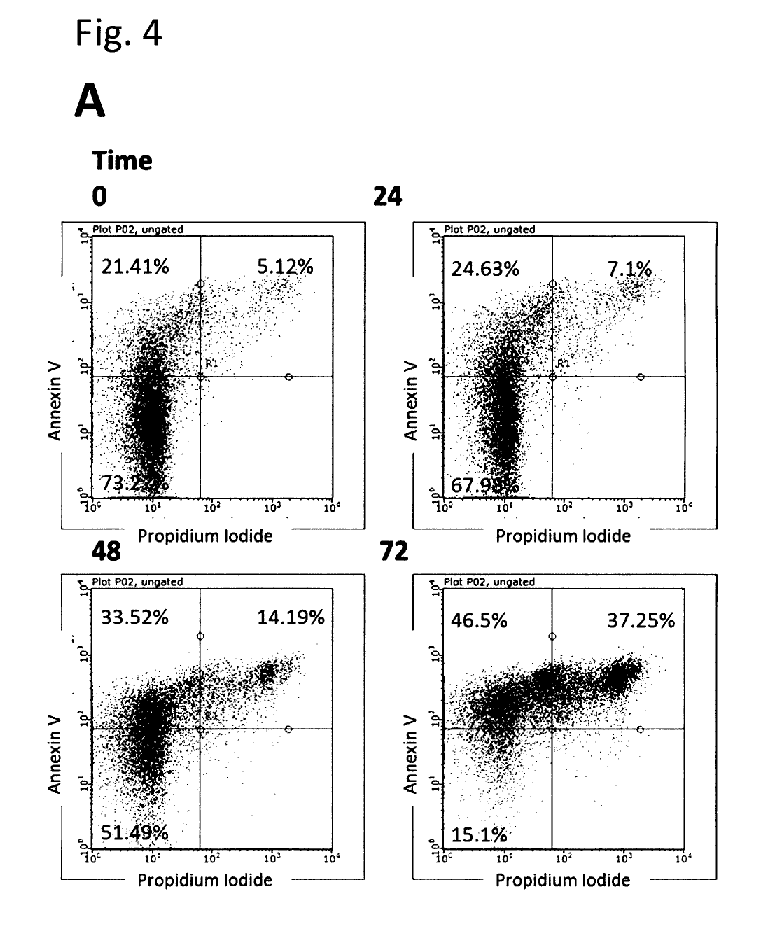

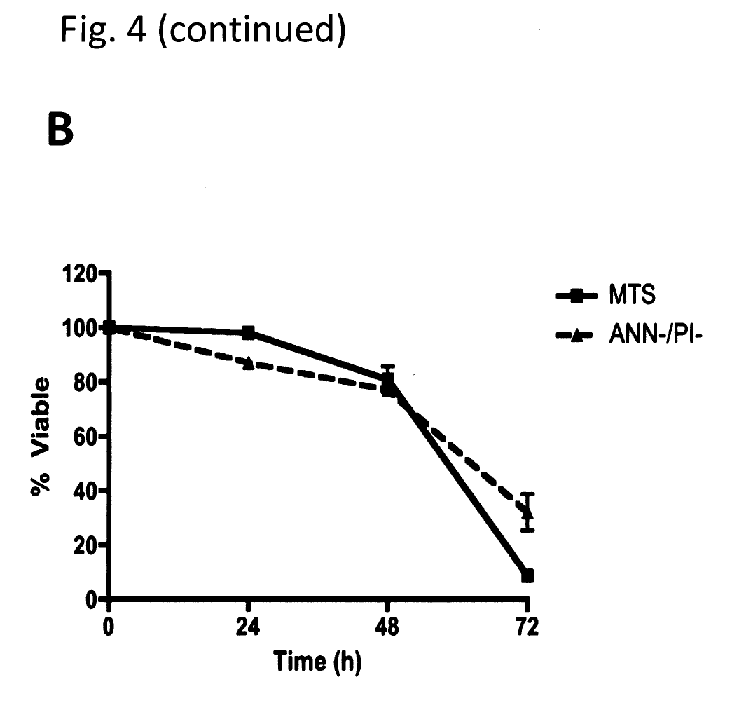

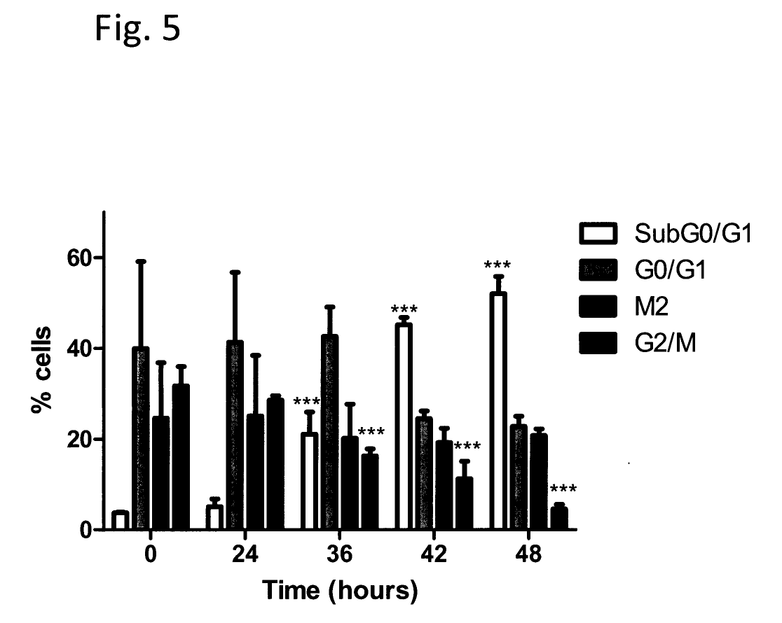

Externalization of phosphatidylserine, an early marker of apoptosis detected by Annexin V, was observed in live cells (i.e., ANN⁺/PI⁻) treated with avocatin B by flow cytometry 48 hours post treatment (FIG. 3A; F_{3,7}=19.09; p<0.05; see FIG. 4 for raw data). This coincided with the occurrence of DNA fragmentation (FIG. 3B; F_{4,14}=171.4; p<0.001), caspase activation (FIG. 3C; F_{3,16}=69.56; p<0.001) and PARP cleavage (FIG. 3D), as measured by cell cycle analysis (see FIG. 5), a caspase activation assay and Western blotting, respectively.

To test whether death was dependent on caspase enzymes avocatin B was co-incubated with the pan-caspase inhibitor Z-VAD-FMK or the caspase-3 specific inhibitor QVD for 72 hours. Both inhibitors only slightly protected from avocatin B-induced death (F_{4,9}=2.714; p<0.01; FIG. 2E). Since cell death can occur independent of caspase enzymes through the release of mitochondria localized proteins such as apoptosis inducing factor (AIF), the presence of AIF was tested for in cytosolic fractions of avocatin B treated TEX cells. However, given that AIF release involves mitochondrial outer membrane permeability and that caspase activation was detected; the presence of cytochrome c, which activates caspase enzymes following its release from the mitochondrial intermembrane space, was simultaneously tested. Cells treated with avocatin B showed an increase in cytoplasmic concentrations of AIF (F_{4,20}=8.211; p<0.001; FIG. 2F) and cytochrome c (F_{4,20}=13.57; p<0.001; FIG. 2F). Therefore, avocatin B induced apoptotic death characterized by the release of the mitochondrial proteins AIF and cytochrome c.

### Avocatin B Inhibits Fatty Acid Oxidation

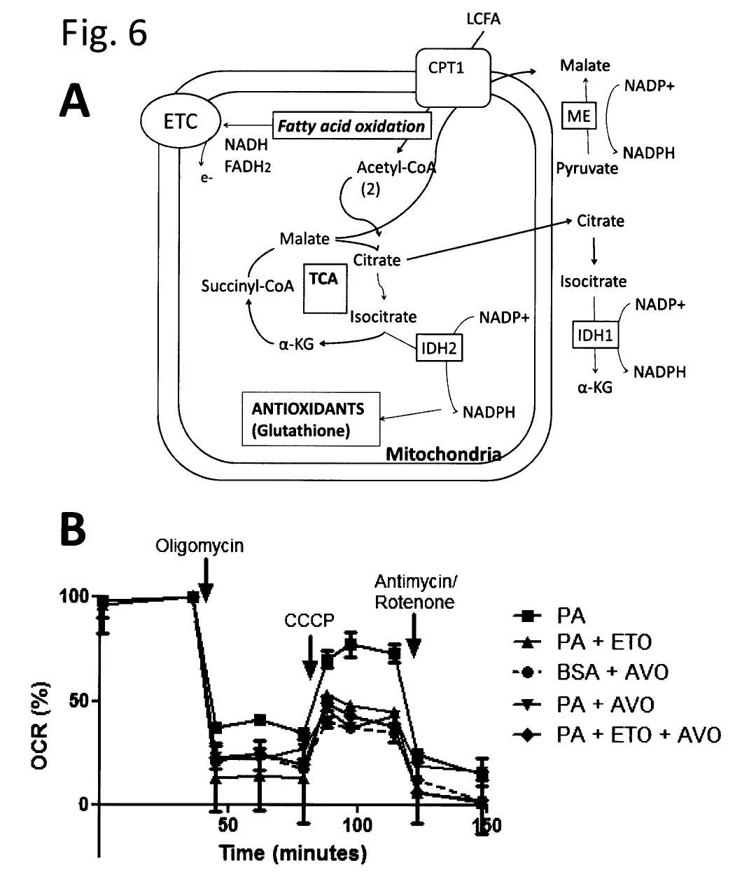

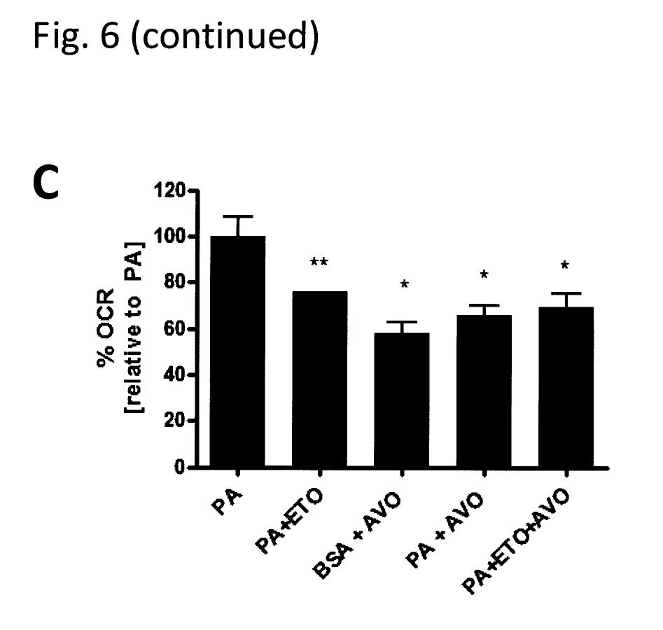

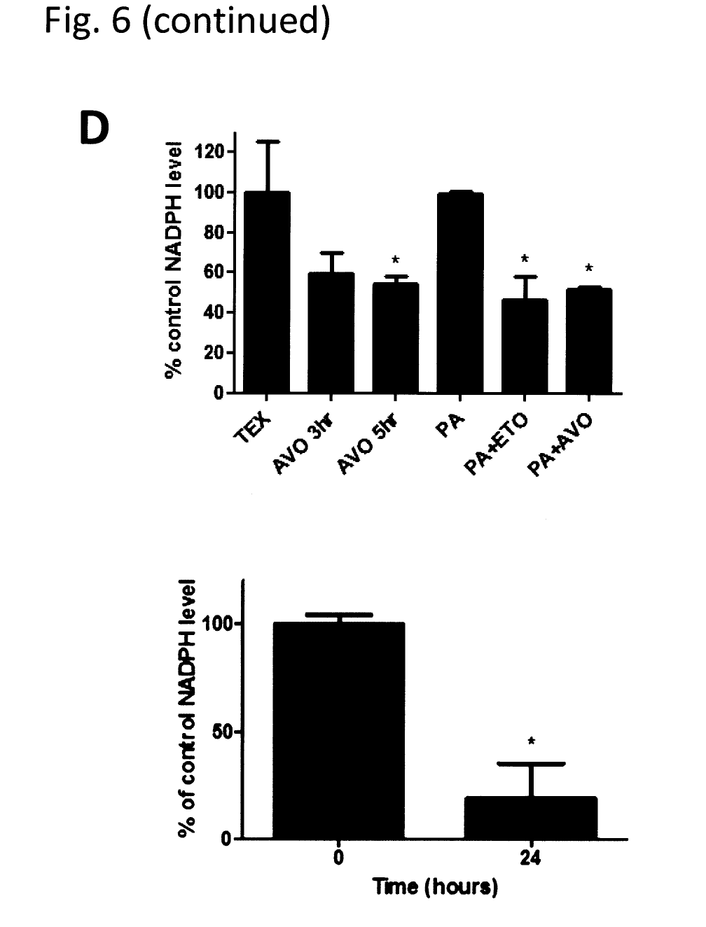

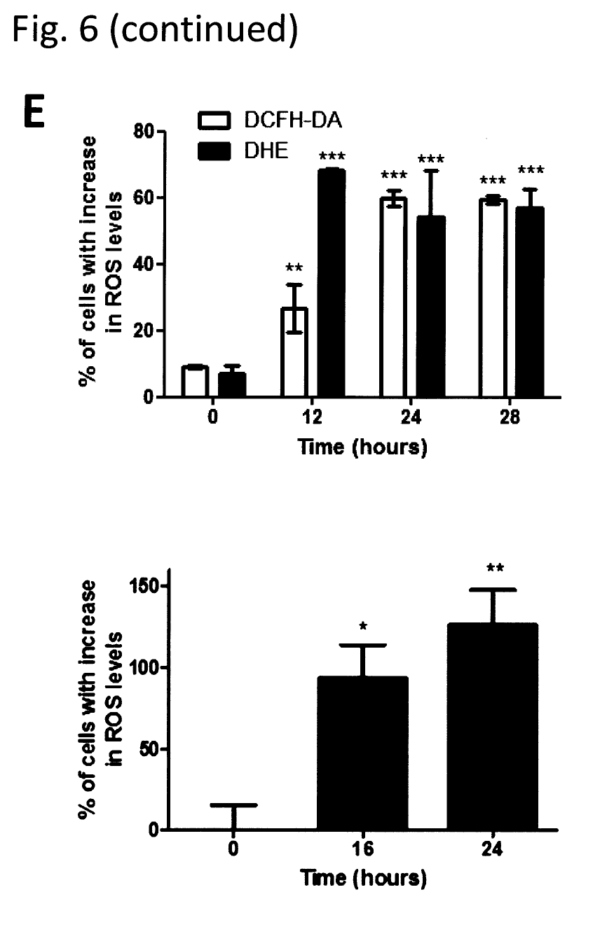

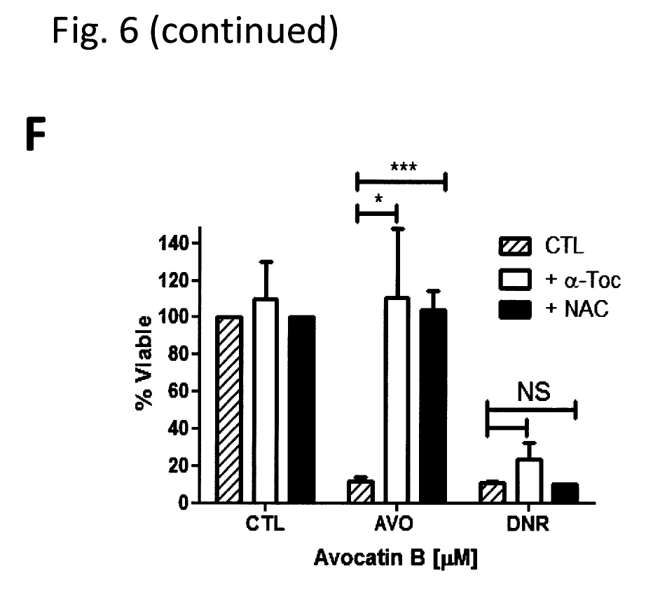

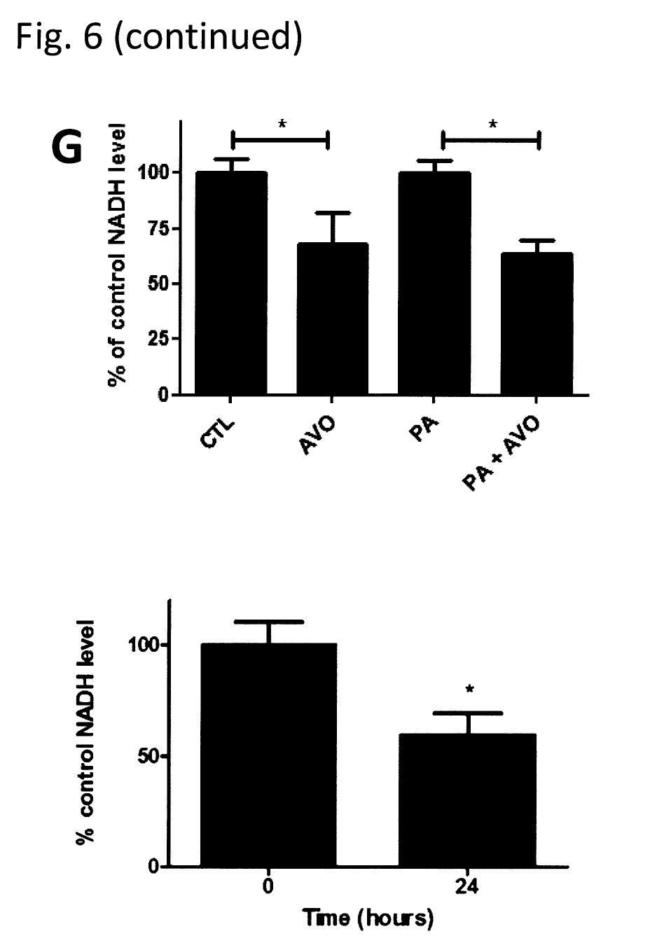

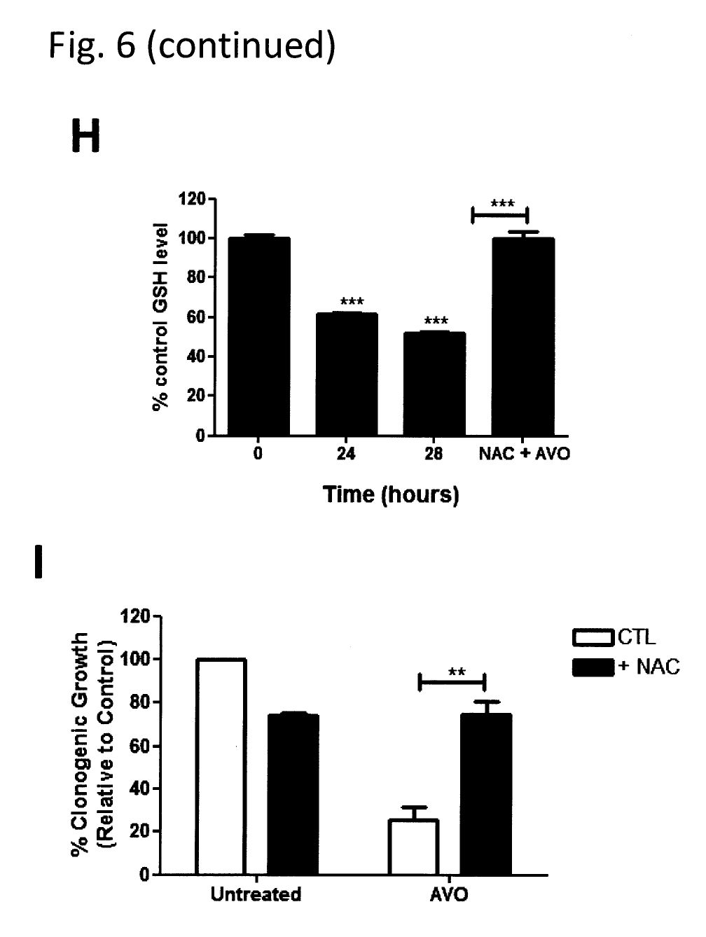

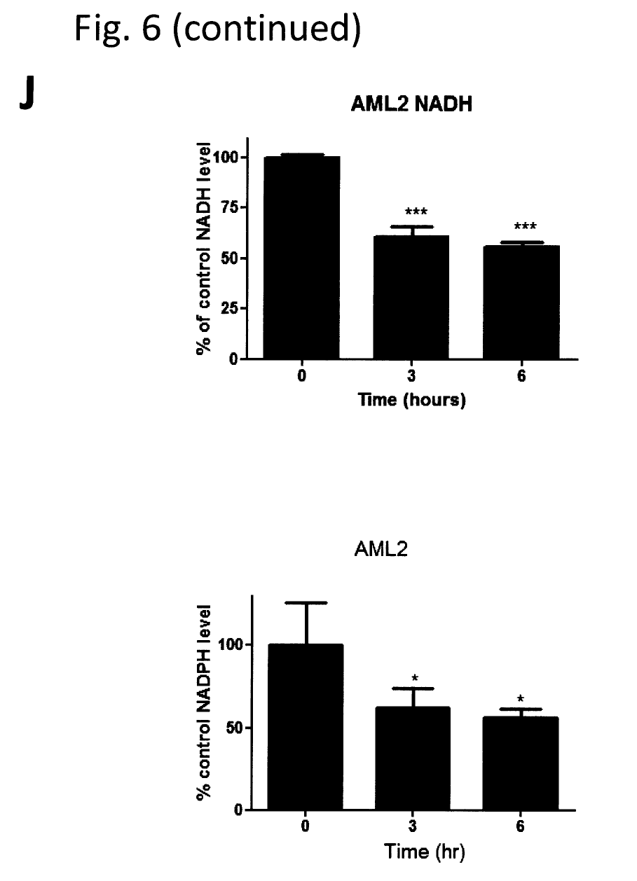

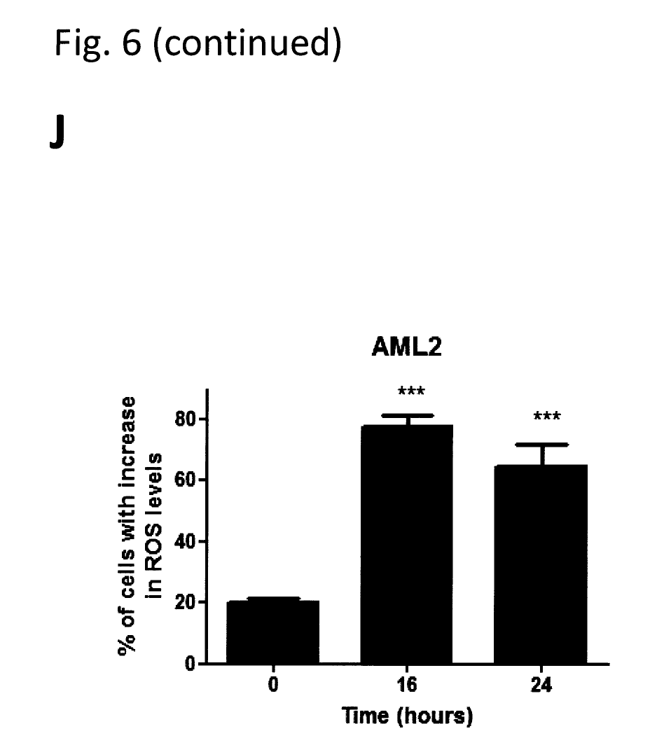

Apoptosis was characterized by the release of mitochondrial proteins following avocatin B treatment. Since avocatin B is a 17-carbon lipid and lipids of that size can enter the mitochondria and undergo fatty acid oxidation after they have been processed by carnitine palmitoyltransferase 1 (CPT1), the impact of avocatin B on fatty acid oxidation was evaluated. Fatty acid oxidation produces acetyl-CoA which enters the TCA cycle to produce NADH, which fuels oxidative phosphorylation, and NADPH, an important co-factor that participates in catabolic processes during cell proliferation²³ and is the precursor of reduced glutathione—an important intracellular and mitochondrial antioxidant^{24,25} (FIG. 6A). To test the effects of avocatin B on fatty acid oxidation, mitochondrial bioenergetics of TEX cells pre-incubated with avocatin B or palmitate in the absence or presence of etomoxir was determined by measuring the change in maximum oxygen consumption following oligomycin and CCCP treatment and prior to the addition of antimycin and rotenone, as described in Abe et al. (2013)¹³. As expected, treatment with palmitate increased the oxygen consumption rate (OCR), consistent with oxidation of exogenous fatty acid substrates and this increase was blocked by treatment with etomoxir, a CPT1 inhibitor (FIGS. 6B&C). Similarly, avocatin B reduced palmitate oxidation demonstrating that avocatin B inhibits the oxidation of exogenous fatty acids (F_{5,17}=40.83; p<0.05; FIGS. 6B&C; arrows indicate the time when oligomycin, CCCP and antimycin/rotenone were added to the cells).

### Inhibiting Fatty Acid Oxidation Results in Reduced NAD, NADPH and GSH and Elevated ROS

Inhibiting fatty acid oxidation can decrease NAD, NADPH and GSH and subsequently decrease antioxidant capabilities¹⁶. Thus, the effect of avocatin B on NAD, NADPH and GSH levels in leukemia cells was tested. Avocatin B (10 μM), similar to etomoxir (100 μM), resulted in a 50% reduction in NADPH, an effect that was observed even in the presence of palmitate (175 μM; F_{9,19}=5.129; p<0.05; FIG. 6D). Similarly, avocatin B decreased NADH and GSH (FIG. 6G: NAD: F_{3,11}=5.145; P<0.05; FIG. 6H: GSH: F_{4,14}=188.9; P<0.001) and decreased NADH and NADPH in treated OCI-AML2 cells which are shown in FIG. 6J.

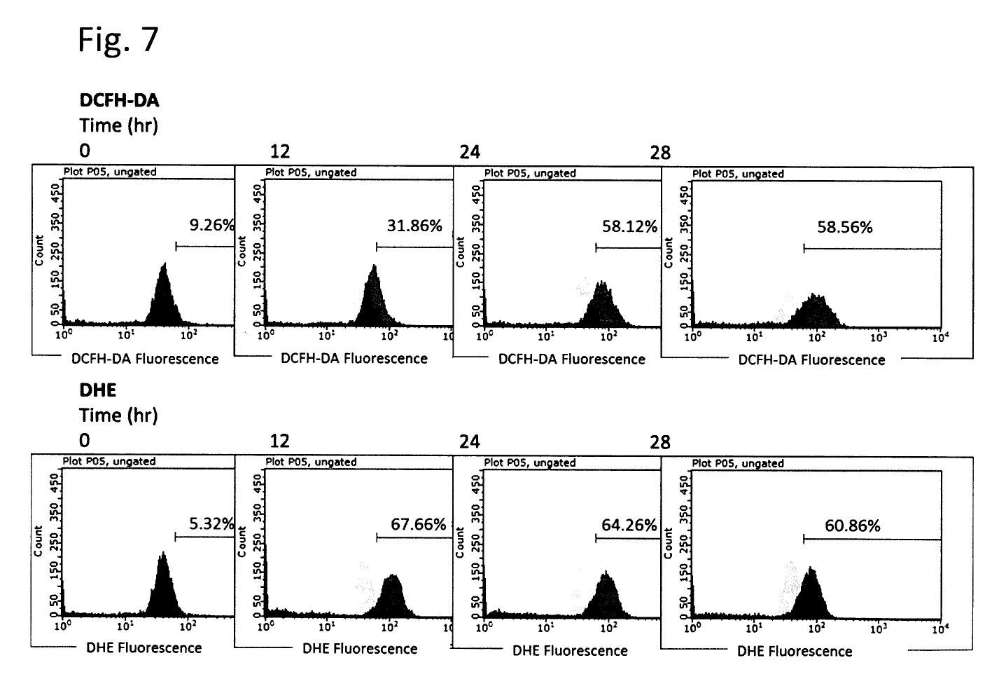

Inhibition of fatty acid oxidation can reduce NADPH and GSH leading to reduced antioxidant capacity, elevated reactive oxygen species (ROS) and cell death¹⁶. ROS levels were tested in avocatin B treated cells using DCFH-DA and DHE, which measure general ROS and superoxide, respectively. TEX or primary AML cells treated with avocatin B had a time-dependent increase in ROS levels as measured by DCFH-DA (F_{5,11}=176.7; p<0.01; FIG. 6E top; see FIG. 7 for histogram data) and DHE (F_{5,11}=36.75; p<0.01; FIG. 6E; see FIG. 7 for histogram data). Similar results were seen with OCI-AML2 as shown in FIG. 6J. To test the importance of ROS in avocatin B-induced death, cells were co-incubated with the antioxidants N-acetylcysteine (NAC) or α-tocopherol (α-Toc). NAC neutralizes ROS through glutathione generation, which is reduced following NADPH depletion¹⁶ and α-tocopherol is a lipid-based antioxidant that accumulates in organelle membranes, particularly mitochondria; to prevent lipid peroxyl radicals formed by ROS induced membrane damage²⁶. Co-incubation with NAC (F_{3,7}=70.55; p<0.05; FIG. 6F and F_{3,10}=70.55, p<0.05; FIG. 6I) or α-tocopherol (F_{3,7}=10.23; p<0.05; FIG. 6F) abolished avocatin B-induced death. Daunorubicin (DNR) was used a negative control, as antioxidants do not protect against its cytotoxicity^{27,28}. Cells were co-incubated with polyethylene glycol-superoxide dismutase (PEG-SOD), an antioxidant that reduces cellular concentrations of the superoxide anion. Co-incubation with PEG-SOD similarly reduced ROS and blocked avocatin B's activity. Together, these results demonstrate that avocatin B reduces levels of NAD, NADPH and GSH and that ROS is functionally important for avocatin B-induced death.

### Mitochondria and CPT1 are Functionally Important for Avocatin B-Induced Death

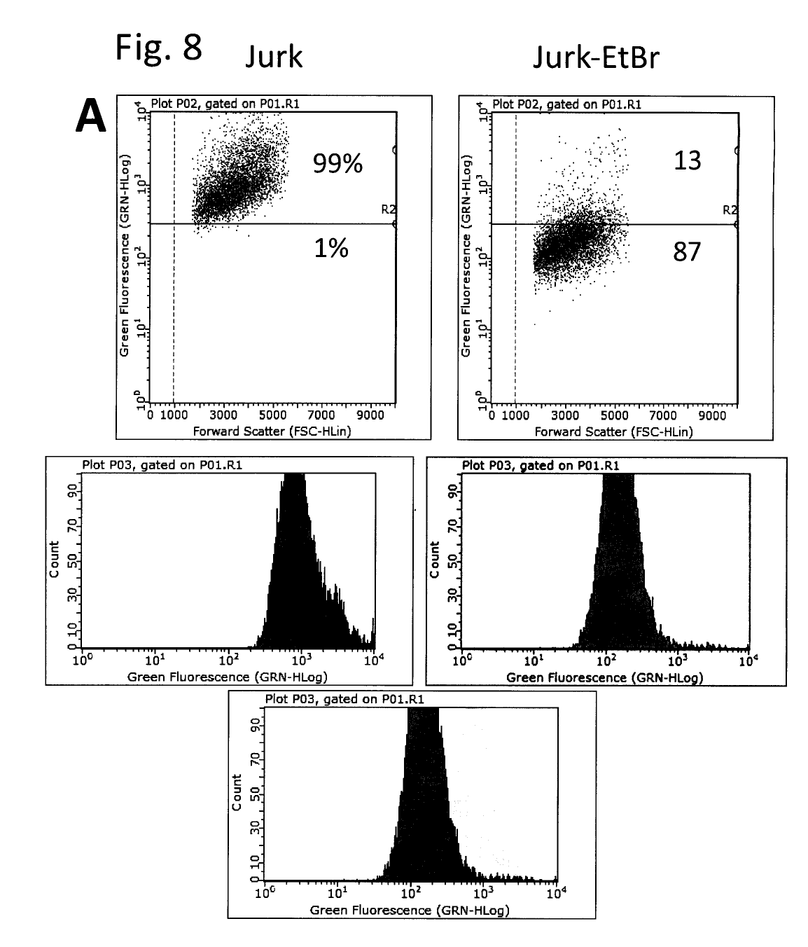

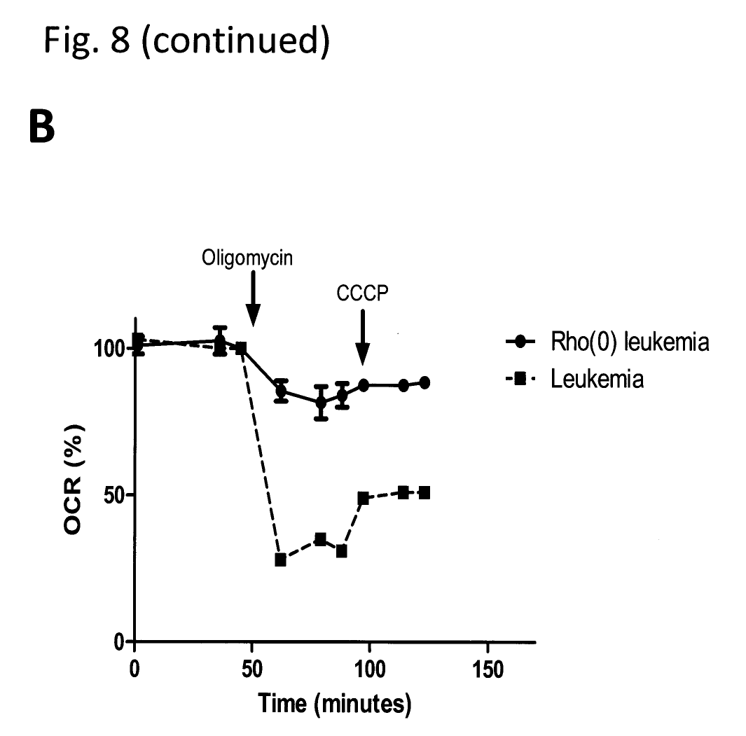

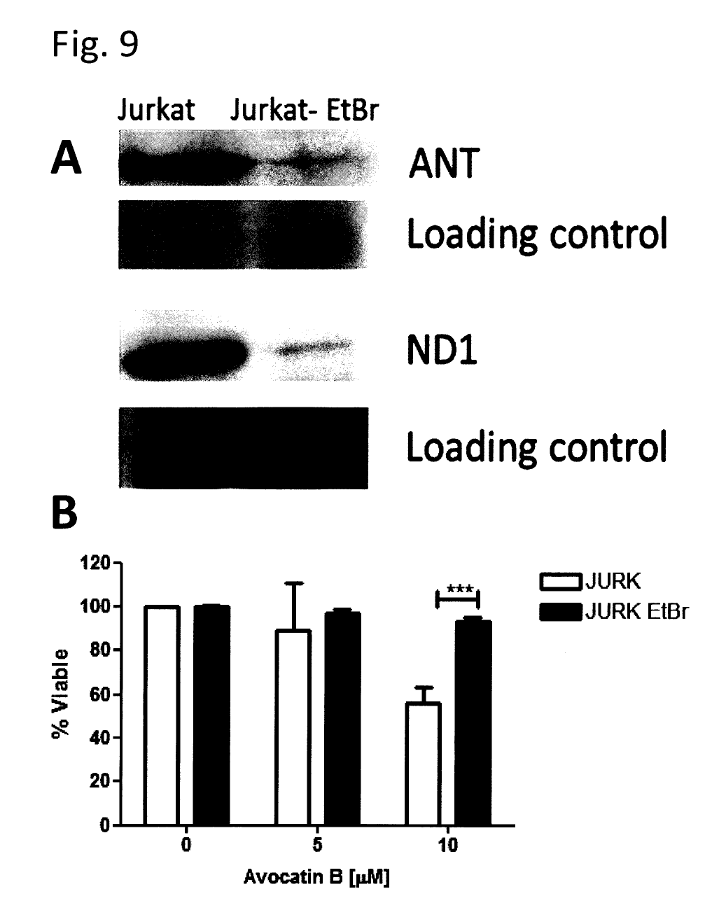

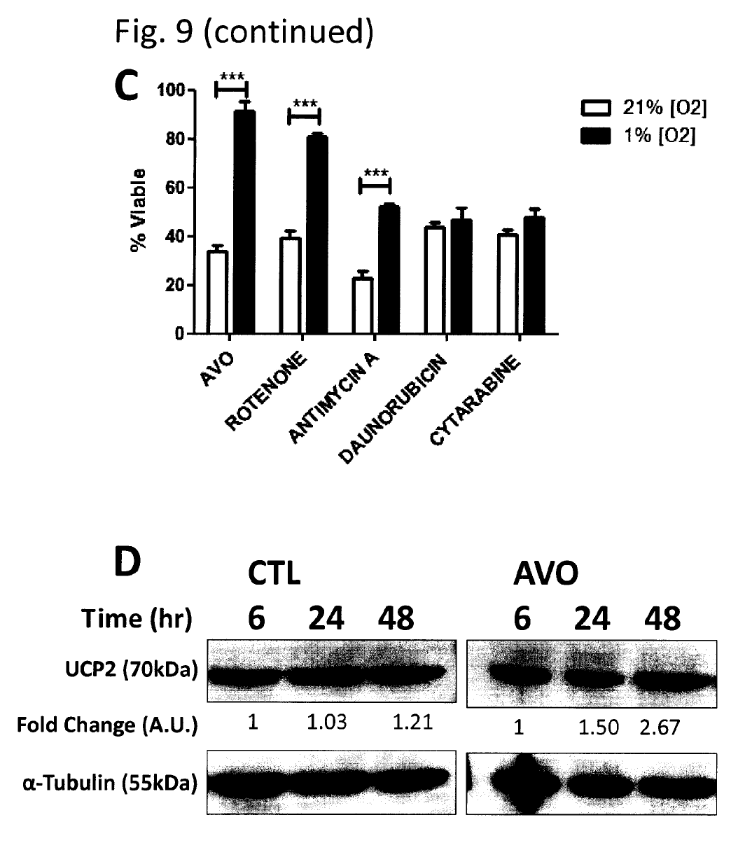

It was demonstrated that avocatin B inhibits fatty acid oxidation and induces apoptosis characterized by the release of mitochondrial proteins cytochrome c and AIF. Since avocatin B is a lipid and leukemia cells possess mitochondrial and metabolic alterations that increase their demand for fatty acid substrates², it was hypothesized that avocatin B's toxicity was related to its localization in mitochondria. To first test avocatin B's reliance on mitochondria for cytotoxicity, leukemia cells lacking functional mitochondria were generated by culturing Jurkat-T cells in media supplemented with 50 ng/ml of ethidium bromide (EtBr), 100 mg/ml sodium pyruvate and 50 μg/ml uridine, as previously described^{29,30}. Following 60 days of passaging only live cells, the presence of mitochondria were tested by flow cytometry following 10-nonyl acridine orange (NAO) staining and by Western blotting for mitochondrial specific proteins ND1 (i.e., complex 1) and adenine nucleotide translocator (ANT). The significant reduction of mitochondria was confirmed, as cells co-cultured in EtBr containing media demonstrated a drastic reduction in NAO staining (FIG. 8A), absence of mitochondrial respiration (FIG. 8B) and a near absence of ND1 and ANT (FIG. 9A). Avocatin B's toxicity was abolished in cells lacking functional mitochondria Jurkat-EtBr cells, as measured by the ANN/PI assay (F_{2,12}=6.509; p<0.001; FIG. 9B). Highlighting the utility of these cells in assessing mitochondrial participation in drug activity, it was previously shown that cells lacking mitochondria were equally sensitive to their mitochondria containing controls when subjected to a compound that activates mitochondria-independent, calpain-mediated apoptosis¹².

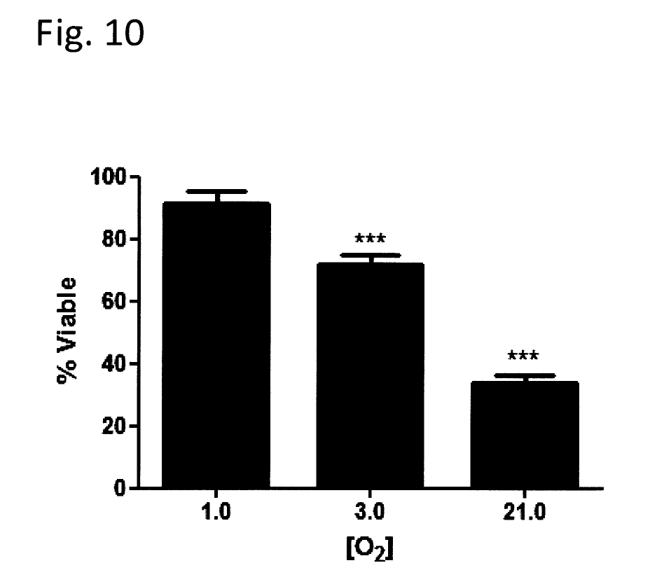

In hypoxic conditions (i.e., reducing oxygen concentrations), ATP production is shifted away from the mitochondrial pathways of oxidative phosphorylation and fatty acid oxidation toward glycolysis.³¹⁻³³ Thus, avocatin B's cytotoxicity in normoxic (21% oxygen) and hypoxic (1% oxygen) conditions was compared. As controls, drugs that directly target mitochondria such as antimycin and rotenone and drugs that do not directly target mitochondria such as cytarabine and daunorubicin were tested. These controls demonstrated that the activity of mitochondria target drugs are reduced in hypoxic conditions whereas non-mitochondria target drugs are unaffected by these conditions. As expected, avocatin B's activity was significantly reduced in conditions in which cellular metabolism is shifted away from mitochondrial pathways (F_{4,24}=98.51; p<0.0001; FIG. 9C). Avocatin B's activity was decreased in reduced oxygen; it did remain active at oxygen concentrations (i.e., 6.1%±1.7%³⁴) found in bone marrow (FIG. 10).

Excessive accumulation of fatty acids within the mitochondria increases the expression of uncoupling proteins (UCP)³⁵. Thus, the expression of UCP2, the leukemia specific UCP³⁶, was measured as an indirect measure of mitochondrial fatty acid accumulation by Western blotting. Treatment with avocatin B increased UCP2 protein expression in leukemia cells (FIG. 9D).

To directly examine whether avocatin B accumulated into mitochondria, LC/MS was performed on mitochondria and cytosolic fractions of avocatin B or vehicle control-treated TEX cells. Fraction purity was confirmed by Western blot analysis of the mitochondria-specific protein ND1. Avocatin B was detected in mitochondrial and cytosolic fractions of avocatin B-treated TEX cells (see Table 2 for estimates). Two peaks [with a mass/charge (m/z) ratio of 285.24242 and 287.25807] were detected, which reflect the nature of avocatin B's two-lipid composition. Importantly, retention times (min) for m/z 285 and 287 were nearly identical between pure compound and the cellular fractions (pure avocatin B: 4.46 and 4.76; mitochondrial fraction: 4.46 and 4.78; cytosolic fraction: 4.46 and 4.78). As expected, avocatin B was not found in vehicle control-treated cells.

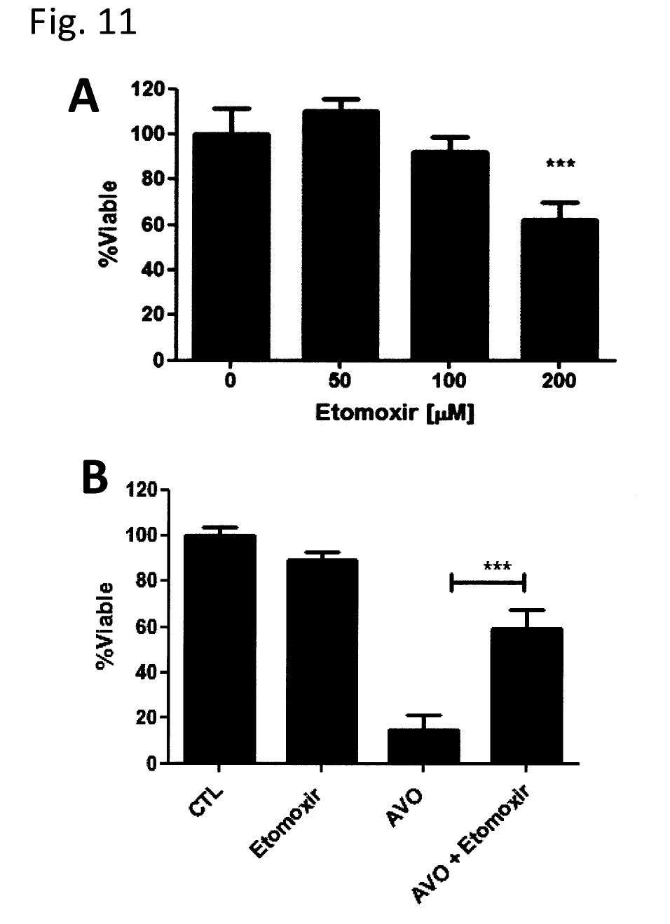

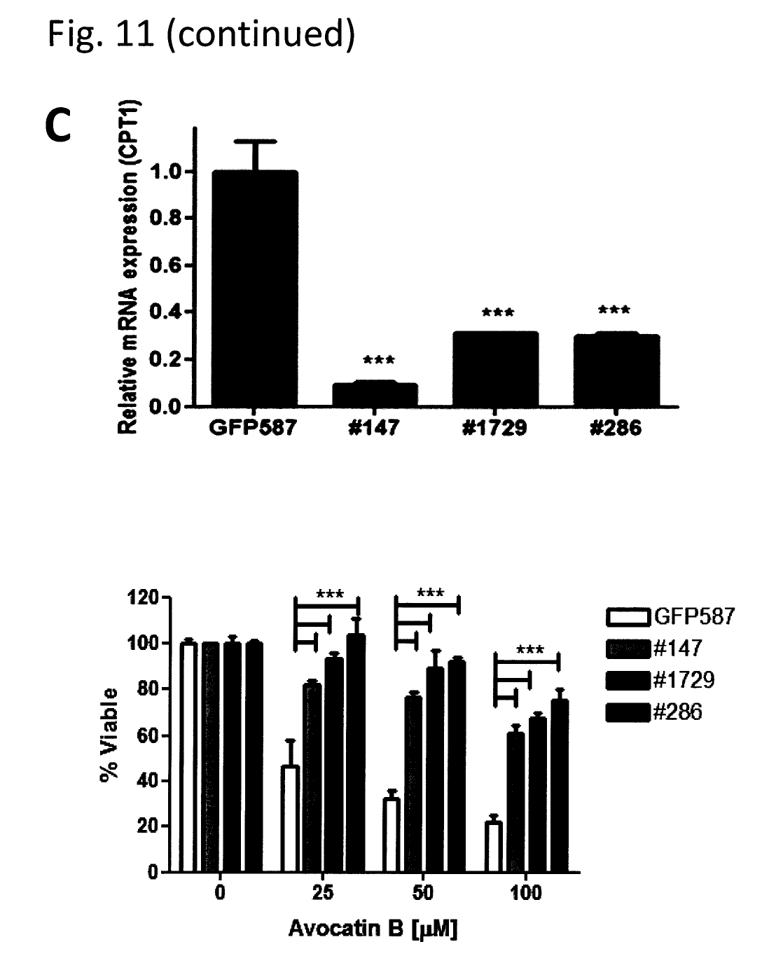

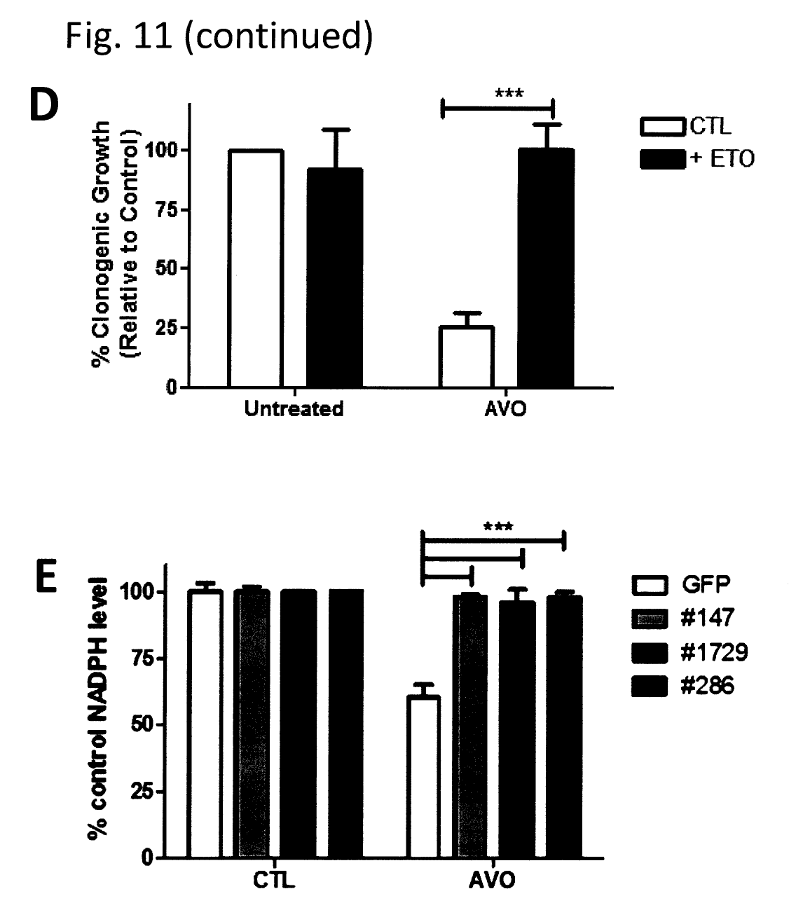

Lipids of 16-20 carbon length enter mitochondria by the activity of CPT1³⁷. To determine the role of CPT1 in avocatin B-induced death, CPT1 activity was chemically blocked with etomoxir and genetically blocked using RNA interference. Etomoxir concentrations that did not reduce viability (100 μM; F_{3,11}=19.81; p<0.001; FIG. 11A), abrogated avocatin B-induced cell death (F_{5,17}=94.45; p<0.001; FIG. 11B) and reductions in clonogenic growth (FIG. 11D; F5,17 ¼ 94.45; P<0.001). As a genetic approach, cells with reduced CPTI gene expression were generated (mRNA: FIG. 11C, top panel; protein: see Shrivani et al. 2014³⁸). CPT1 knockdown cells were significantly less sensitive to avocatin B (F_{9,32}=23.73; p<0.001; FIG. 11C, bottom panel) and were insensitive to avocatin B-induced reduction of NADPH (FIG. 11E; F3,16=65.04; p<0.001). Together, these results suggest that avocatin B is a lipid that localizes to the mitochondria and impairs fatty acid oxidation.

### Discussion

A screen of a natural health product library identified avocatin B as a novel anti-AML agent. In vitro and pre-clinical functional studies demonstrated that it induced selective toxicity toward leukemia and leukemia stem cells with no toxicity toward normal cells. Mechanistically, a strategy is highlighted to induce selective leukemia cell death where mitochondrial localization of avocatin B inhibits fatty acid oxidation leading to reduced levels of NADPH and elevated ROS leading to apoptosi cell death.

Avocatin B targets leukemia over normal cells. It is proposed that this specificity is related to the leukemia cell's altered mitochondrial characteristics, as a number of observations suggest avocatin B localizes in mitochondria. For example, (1) avocatin B accumulates in leukemia cell mitochondria (e.g. demonstrated using LC/MS), (2) cells with significantly reduced mitochondria or (3) lacking the enzyme that facilitates mitochondrial lipid transport, CPT1, are insensitive to avocatin B; (4) chemical treatment with etomoxir, a CPT1 inhibitor, blocked avocatin B's activity; (5) CPT1 only facilitates entry of lipids of avocatin B's size into mitochondria (e.g., 16-20 carbons³⁷; avocatin B:17 carbons²¹); and (5) UCP2 levels are increased following avocatin B treatment. Leukemia cells contain higher mitochondrial mass¹ and greater demand for fatty acid substrates for metabolic activity² compared to normal hematopoietic cells.² Thus, given the leukemia cell's mitochondrial phenotype, it is proposed that avocatin B accumulates with greater concentration in leukemia over normal cells, thus conferring its increased toxicity toward leukemia cells.

Inhibition of fatty acid oxidation by avocatin B resulted in ROS-induced apoptosis. Apoptosis was mediated by the mitochondrial proteins cytochrome c and AIF, which are commonly released following ROS-induced increases in mitochondrial outer membrane permeability^{39,40}. Inhibiting fatty acid oxidation by blocking CPT1 with etomoxir resulted in ROS-dependent death of glioma cells caused by reduced concentrations of intracellular antioxidants attributed to decreased NADPH¹⁶. Similarly, it was demonstrated that avocatin B-induced inhibition of fatty acid oxidation reduced NADPH levels and that antioxidant supplementation rescued cells from death. NADPH is utilized for catabolic processes in proliferating cells and is the precursor of reduced glutathione, which counteracts the detrimental effects of ROS^{23,41}. The observed NADPH decrease (t=5 h; FIG. 5D) preceded ROS elevation (t=12 h; FIG. 5E), further confirming the relationship between inhibition of fatty acid oxidation, NADPH and ROS-dependent leukemia cell death. Avocatin B accumulated in mitochondria and inhibited fatty acid oxidation and reduced NADPH at 10 μM whereas other studies used etomoxir, which blocks fatty acid entry into mitochondria and reduces NADPH, at 100 μM² or 1000 μM¹⁶. Together, these results point to a mechanism where avocatin B enters the mitochondria and potently inhibits fatty acid oxidation resulting in reduced NADPH and GSH leading to elevated ROS and apoptotic cell death.

Avocatin B is a mixture of 17-carbon lipids derived from methanol extracted avocado pear seeds (Persea gratissima)²⁰. Odd-numbered carbons are rare, not produced endogenously and obtained only from dietary sources^{42,43}. Moreover, they are not efficiently or preferentially oxidized. For example, mice fed diets containing radiolabelled odd and even-numbered fatty acids only accumulate odd-numbered fatty acids in adipose tissue (i.e., C15 and 17)⁴⁴; odd-numbered fatty acids show consistent adipose accumulation^{42,45,46}. In humans, lipids of 13, 15 and 17 carbon lengths are used as serum and adipose tissue biomarkers of dietary fat intake, as these fatty acids are more slowly catabolized compared to even-numbered fatty acids^{43,47}. Although they undergo the same pathway of oxidation, the terminal step of odd-numbered fatty acid oxidation produces 1 acetyl-coA and 1 propionyl-CoA molecule whereas even-numbered fatty acids produce 2 acetyl-coA molecules⁴⁸. Propionyl-CoA can then be converted to methylmalonyl-CoA by propinyl-CoA carboxylase and vitamin B12, at the expense of 1 ATP, which is in turn converted to succinyl-CoA that can enter the TCA cycle⁴⁶. Since this alternate pathway requires energy and delays overall ATP production, the decreased metabolic activity (i.e., reduced acetyl-CoA production and/or decreased entry of fatty acid byproducts into the TCA cycle) likely explains the observed decrease in NADPH. As such, reduced NADPH not only results in elevated ROS but also indicates a decrease in overall metabolic activity. Thus, a pathway by which fatty acid oxidation can be inhibited in leukemia cells is by the odd-numbered carbon lipid, avocatin B stalling or rendering less efficient the fatty acid oxidation pathway. This highlights a strategy to induce selective leukemia cell death by which preferential mitochondrial localization of avocatin B reduces leukemia cell metabolism and NADPH to increase ROS resulting in cell death.

Initial assessment of avocatin B's physicochemical properties suggests favorable tissue distribution. In particular, it possesses a high estimated partition coefficient (Log P=8.921) indicating that it will accumulate in lipid-rich tissues such as adipose tissue and bone marrow. Given that LSCs reside in bone marrow, this could significantly enhance avocatin B's therapeutic efficacy.

TABLE 1a
AML patient sample details used for annexin/PI (ANN/PI)

| Label | CD34 | |
|---|---|---|
| # | (%) | Interpretation |
| AML1 | 77 | 43~44, XX, dic(3; 12)(p13; p11.2), −5, −7, |
| | | +del(11)(q13), ?dup(18)(q21q22), del(20)(q13.1), |
| | | −22, +mar[cp10] |
| AML2 | 88 | 44~48, XX, del(5)(q13q33), i(9)(p10), −18, |
| | | add(20)(q13.3), +mar[cp17]/46, XX[3] |
| AML3 | 87 | 46, XX, t(2; 8)(p13; q22)[20].ish t(2; 8)(MYC+; |
| | | MYC−) |
| AML4 | 1 | NPM, Flt3-TKD |
| AML5 | 60 | ** |
| AML5 | 77 | Del 5q |

** Denotes unknown/not tested

TABLE 1b
AML patient sample details used for colony formation assays

| Label | CD34 | |
|---|---|---|
| # | (%) | Interpretation |
| AML6 | ** | 43, XY, del(5)(q13q33), −7, t(9; 12)(q12; q12), |
| | | der(16)t(16; 17)(q12.1; q21), −17, −18[4]/44, idem, |
| | | +8[2]/46, XY[3] |
| AML7 | ** | 46, XX, t(9; 11)(p22; q23)[20] |
| AML8 | ** | 46, XY, del(5)(q22q31)[20] |

TABLE 2
Estimated concentration of Avocatin B in Mitochondrial
and Cytosolic Fractions of Treated Cells

| | | Concentration |
|---|---|---|
| Treatment | Fraction | [ng/mL] |
| Control | Cytosolic | <LOD |
| | Mitochondrial | <LOD |
| | Cytosolic | 1253.6 |
| AVO treated [10 μM] | Mitochondrial | 882.7 |

### Example 2

AML patient cells were injected into left femurs of NOD/SCID mice. Mice were treated with avocatin B (100 mg/kg bw; 18 times in 4 weeks). Mice were sacrificed and right femurs excised and bone marrow assessed for human myeloid cells (CD45+, CD19−, CD33+). Five of 9 mice treated with avocatin B showed a greater than 50% average decrease in the percent of CD45+, CD19−, CD33+ cells relative to control treated mice (n=10). Avocatin B was prepared in PBS+ Tween and mice were injected intraperitoneally.

### Example 3

AML patient cells were injected into left femurs of NOD/SCID mice. Mice were treated with avocatin B (100 mg/kg bw; 18 times in 4 weeks). Mice were sacrificed and toxicity blood markers were measured in blood at time of sacrifice. Bilirubin, a marker of red blood cell lysis was similar in control and treated mice. Creatine which is a liver function marker was also similar between control and treated mice. Aspartate transaminase and alkaline phosphatase were decreased in treated mice.

### Example 4

Leukemic cell lines were incubated with Avocatin B for 72 hours and assessed by MTS assay. Jurkat, K562 (CML), HL60 (APML) in addition to AML cell lines TEX, AML2 and KG1A showed toxicity to avocatin B. U937 cells showed toxicity only with high concentrations of avocatin B.

### Example 5

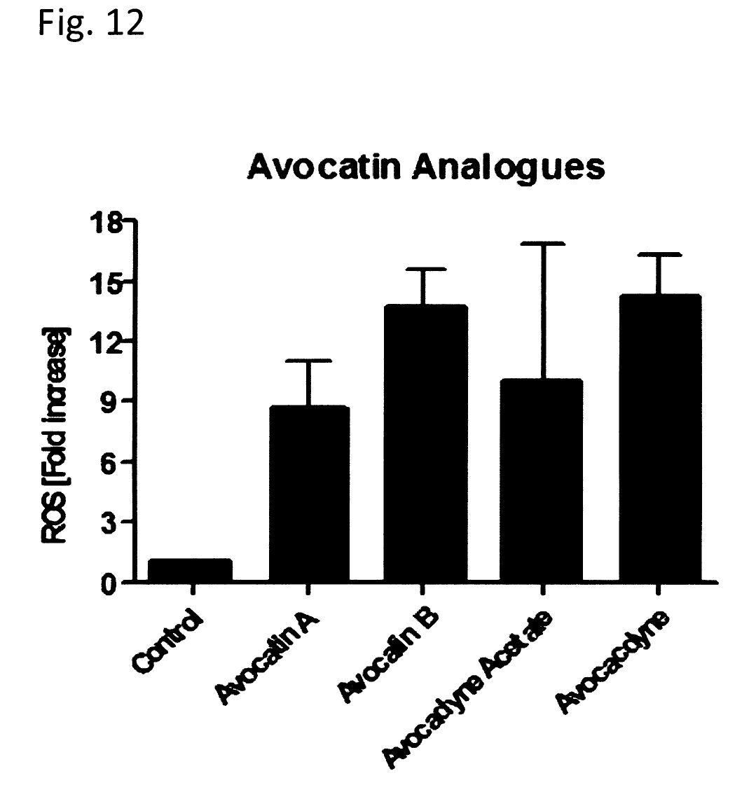

TEX cells were treated with avocatin analogues and measured for ROS activity as described in Example 1. As demonstrated in FIG. 12, avocadyne, avocadyne acetate and avocatin A induce ROS activity.

### Example 6

Avocadyne, avocadene and avocatin B were tested for their toxicity to leukemic cells using TEX cells. The TEX cells were treated with increasing concentrations of each lipid (up to 10 micromolar) and subsequently stained with propidium iodide (PI), which stains only dead cells. The percent viability was measured using flow cytometry gating on PI positive and PI negative cells. Tex cells treated with avocadyne showed the greatest amount of cell death (i.e., PI positive) followed by cells with avocatin B. Tex cells treated with avocadene, showed the least amount of cell death.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession numbers provided herein including for example accession numbers and/or biomarker sequences (e.g. protein and/or nucleic acid) provided in the Tables or elsewhere, are incorporated by reference in its entirely

### CITATIONS FOR REFERENCES REFERRED TO IN THE SPECIFICATION

1. Skrtic M, Sriskanthadevan S, Jhas B, et al. Inhibition of mitochondrial translation as a therapeutic strategy for human acute myeloid leukemia. Cancer cell. Nov. 15, 2011; 20(5):674-688.
2. Samudio I, Harmancey R, Fiegl M, et al. Pharmacologic inhibition of fatty acid oxidation sensitizes human leukemia cells to apoptosis induction. The Journal of clinical investigation. January 2010; 120(1):142-156.
3. Shipley J L, Butera J N. Acute myelogenous leukemia. Experimental hematology. June 2009; 37(6):649-658.
4. Lowenberg B, Downing J R, Burnett A. Acute myeloid leukemia. The New England journal of medicine. Sep. 30, 1999; 341(14):1051-1062.
5. McDermott S P, Eppert K, Notta F, et al. A small molecule screening strategy with validation on human leukemia stem cells uncovers the therapeutic efficacy of kinetin riboside. Blood. Feb. 2, 2012; 119(5):1200-1207.
6. Spagnuolo P A, Hurren R, Gronda M, et al. Inhibition of intracellular dipeptidyl peptidases 8 and 9 enhances parthenolide's anti-leukemic activity. Leukemia: official journal of the Leukemia Society of America, Leukemia Research Fund, U.K. Jan. 15, 2013.
7. Spagnuolo P A, Hu J, Hurren R, et al. The antihelmintic flubendazole inhibits microtubule function through a mechanism distinct from Vinca alkaloids and displays preclinical activity in leukemia and myeloma. Blood. Jun. 10, 2010; 115(23):4824-4833.
8. Sukhai M A, Prabha S, Hurren R, et al. Lysosomal disruption preferentially targets acute myeloid leukemia cells and progenitors. The Journal of clinical investigation. Dec. 3, 2012.
9. Sachlos E, Risueno R M, Laronde S, et al. Identification of drugs including a dopamine receptor antagonist that selectively target cancer stem cells. Cell. Jun. 8, 2012; 149(6):1284-1297.
10. Chou T C, Talalay P. Quantitative analysis of dose-effect relationships: the combined effects of multiple drugs or enzyme inhibitors. Advances in enzyme regulation. 1984; 22:27-55.
11. Chou T C. Preclinical versus clinical drug combination studies. Leukemia & lymphoma. November 2008; 49(11):2059-2080.
12. Angka L, Lee E A, Rota S G, et al. Glucopsychosine increases cytosolic calcium to induce calpain-mediated apoptosis of acute myeloid leukemia cells. Cancer letters. Mar. 12, 2014.
13. Abe Y, Sakairi T, Beeson C, Kopp J B. TGF-beta1 stimulates mitochondrial oxidative phosphorylation and generation of reactive oxygen species in cultured mouse podocytes, mediated in part by the mTOR pathway. American journal of physiology. Renal physiology. Nov. 15, 2013; 305(10):F1477-1490.
14. Dam A D, Mitchell A S, Rush J W, Quadrilatero J. Elevated skeletal muscle apoptotic signaling following glutathione depletion. Apoptosis: an international journal on programmed cell death. January 2012; 17(1):48-60.
15. Owusu-Ansah E, Yavari A, Banerjee U. A protocol for _in vivo_{—} detection of reactive oxygen species. 2008.
16. Pike L S, Smift A L, Croteau N J, Ferrick D A, Wu M. Inhibition of fatty acid oxidation by etomoxir impairs NADPH production and increases reactive oxygen species resulting in ATP depletion and cell death in human glioblastoma cells. Biochimica et biophysica acta. June 2011; 1807(6):726-734.
17. Christensen M E, Jansen E S, Sanchez W, Waterhouse N J. Flow cytometry based assays for the measurement of apoptosis-associated mitochondrial membrane depolarisation and cytochrome c release. Methods. Jun. 1, 2013; 61(2):138-145.
18. Waterhouse N J, Trapani J A. A new quantitative assay for cytochrome c release in apoptotic cells. Cell death and differentiation. July 2003; 10(7):853-855.
19. Warner J K, Wang J C, Takenaka K, et al. Direct evidence for cooperating genetic events in the leukemic transformation of normal human hematopoietic cells. Leukemia: official journal of the Leukemia Society of America, Leukemia Research Fund, U.K. October 2005; 19(10):1794-1805.
20. Alves H M C D, Falshaw C P, Godtfredsen W O, Ollis, W D The Avocatins—a New Class of Natural Products. Annals of the Brazilian Academy of Sciences. 1970; 42:4.
21. ChemBank. 2014; http://chembank.broadinstitute.org/chemistry/viewMolecule.htm; jsessionid=9CC037B2065BF DE714299282DD50A255?cbid=3198534.
22. Konopleva M, Watt J, Contractor R, et al. Mechanisms of antileukemic activity of the novel Bcl-2 homology domain-3 mimetic GX15-070 (obatoclax). Cancer research. May 1, 2008; 68(9):3413-3420.
23. Carracedo A, Cantley L C, Pandolfi P P. Cancer metabolism: fatty acid oxidation in the limelight. Nature reviews. Cancer. April 2013; 13(4):227-232.
24. Heiden M G V, Cantley L C, Thompson C B. Understanding the Warburg Effect: The Metabolic Requirements of Cell Proliferation. Science. May 22, 2009; 324(5930):1029-1033.
25. Kirsch M, De Groot H. NAD(P)H, a directly operating antioxidant? FASEB journal: official publication of the Federation of American Societies for Experimental Biology. July 2001; 15(9):1569-1574.
26. Godbout J P, Berg B M, Kelley K W, Johnson R W. alpha-Tocopherol reduces lipopolysaccharide-induced peroxide radical formation and interleukin-6 secretion in primary murine microglia and in brain. Journal of neuroimmunology. April 2004; 149(1-2):101-109.
27. Bejar D E E, Fulbright J M, Corrales-Medina F F, Irwin M E, Johnson B, Chandra J. Uncovering Cardioprotective Strategies For Anthracycline Therapy By Analysis Of Redox and Apoptotic Effects. Blood. Nov. 15, 2013; 122(21).
28. Vejpongsa P, Yeh E T H. Topoisomerase 2 beta: A Promising Molecular Target for Primary Prevention of Anthracycline-Induced Cardiotoxicity. Clin Pharmacol Ther. January 2014; 95(1):45-52.
29. Desjardins P, Frost E, Morais R. Ethidium bromide-induced loss of mitochondrial DNA from primary chicken embryo fibroblasts. Molecular and cellular biology. May 1985; 5(5):1163-1169.
30. Hashiguchi K, Zhang-Akiyama Q M. Establishment of human cell lines lacking mitochondrial DNA. Methods in molecular biology. 2009; 554:383-391.
31. Belanger A J, Luo Z, Vincent K A, et al. Hypoxia-inducible factor 1 mediates hypoxia-induced cardiomyocyte lipid accumulation by reducing the DNA binding activity of peroxisome proliferator-activated receptor alpha/retinoid X receptor. Biochemical and biophysical research communications. Dec. 21, 2007; 364(3):567-572.
32. Cairns R A, Harris I S, Mak T W. Regulation of cancer cell metabolism. Nature reviews. Cancer. February 2011; 11(2):85-95.
33. Ito K, Suda T. Metabolic requirements for the maintenance of self-renewing stem cells. Nature reviews. Molecular cell biology. April 2014; 15(4):243-256.
34. Fiegl M, Samudio I, Clise-Dwyer K, Burks J K, Mnjoyan Z, Andreeff M. CXCR4 expression and biologic activity in acute myeloid leukemia are dependent on oxygen partial pressure. Blood. Feb. 12, 2009; 113(7):1504-1512.
35. Schrauwen P, Hoeks J, Schaart G, et al. Uncoupling protein 3 as a mitochondrial fatty acid anion exporter. FASEB journal: official publication of the Federation of American Societies for Experimental Biology. December 2003; 17(15):2272-2274.
36. Samudio I, Fiegl M, McQueen T, Clise-Dwyer K, Andreeff M. The warburg effect in leukemia-stroma cocultures is mediated by mitochondrial uncoupling associated with uncoupling protein 2 activation. Cancer research. Jul. 1, 2008; 68(13):5198-5205.
37. Reddy J K, Hashimoto T. Peroxisomal beta-oxidation and peroxisome proliferator-activated receptor alpha: an adaptive metabolic system. Annual review of nutrition. 2001; 21:193-230.
38. Shrivani Sriskanthadevan D V J, Timothy E. Chung, Swayam Prabha, Wei Xu, Marko Skrtic, Bozhena Jhas, Rose Hurren, Marcela Gronda, Xiaoming Wang, Yulia Jitkova, Mahadeo A. Sukhai, Feng-Hsu Lin, Neil Maclean, Rob Laister, Carolyn A. Goard, Peter J. Mullen, Stephanie Xie, Linda Z. Penn, Ian Rogers, John E. Dick, Mark D. Minden, Aaron D. Schimmer** AML cells have low spare reserve capacity in their respiratory chain that renders them susceptible to oxidative metabolic stress. Blood. 2014; Submitted.
39. Tomasello F, Messina A, Lartigue L, et al. Outer membrane VDAC1 controls permeability transition of the inner mitochondrial membrane in cellulo during stress-induced apoptosis. Cell research. December 2009; 19(12):1363-1376.
40. Kroemer G, Galluzzi L, Brenner C. Mitochondrial membrane permeabilization in cell death. Physiological reviews. January 2007; 87(1):99-163.
41. Vander Heiden M G, Cantley L C, Thompson C B. Understanding the Warburg effect: the metabolic requirements of cell proliferation. Science. May 22, 2009; 324(5930):1029-1033.
42. Campbell R G, Hashim S A. Deposition in adipose tissue and transport of odd-numbered fatty acids. The American journal of physiology. December 1969; 217(6):1614-1618.
43. Wolk A, Furuheim M, Vessby B. Fatty acid composition of adipose tissue and serum lipids are valid biological markers of dairy fat intake in men. The Journal of nutrition. March 2001; 131(3):828-833.
44. Gotoh N, Moroda K, Watanabe H, et al. Metabolism of odd-numbered fatty acids and even-numbered fatty acids in mouse. Journal of oleo science. 2008; 57(5):293-299.
45. Pi-Sunyer F X. Rats enriched with odd-carbon fatty acids. Effect of prolonged starvation on liver glycogen and serum lipids, glucose and insulin. Diabetes. April 1971; 20(4):200-205.
46. Vanitallie T B, Khachadurian A K. Rats enriched with odd-carbon fatty acids: maintenance of liver glycogen during starvation. Science. Aug. 22, 1969; 165(3895):811-813.
47. Klein R A, Halliday D, Pittet P G. The use of 13-methyltetradecanoic acid as an indicator of adipose tissue turnover. Lipids. August 1980; 15(8):572-579.
48. Berg J M T J, Stryer L. Biochemistry. 5th edition. Certain Fatty Acids Require Additional Steps for Degradation. New York: W H Freeman; 2002: http://www.ncbi.nlm.nih.gov/books/NBK22387/.
49. Guzman M L, Rossi R M, Karnischky L, et al. The sesquiterpene lactone parthenolide induces apoptosis of human acute myelogenous leukemia stem and progenitor cells. Blood. Jun. 1, 2005; 105(11):4163-4169.

1. A method of treating a leukemia comprising administering to a subject in need thereof a therapeutically effective amount of a compound of Formula (I) and/or (II) having the structure: 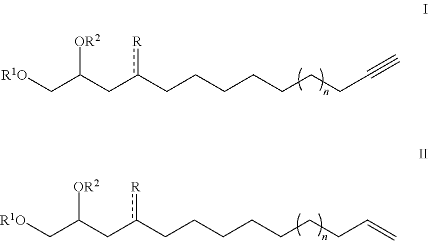
wherein: [symbol]
represents a single or a double bond;
R is OH when C [symbol] R is C—R, and R is O when C [symbol] R is C═R;
n is 1, 3, 5 or 7; and
R¹ and R² are independently hydrogen or acetyl,
and/or isomers, stereoisomers or solvates thereof and/or mixtures thereof, preferably wherein the compound is a compound of Formula (I), more preferably wherein the compound comprises a n=5, and even more wherein compound is avocadyne, avocadyne acetate and/or avocadynone acetate or a mixture comprising avocadyne, avocadyne acetate and/or avocadynone acetate.

2. (canceled)

3. The method according to claim 1, wherein the compound of Formula (I) and/or (II) and/isomers, stereoisomers or solvates thereof and/or mixtures thereof inhibits mitochondrial fatty acid oxidation in a leukemia cell or decreases levels of nicotinamide adenine dinucleotide phosphate (NADPH), NADH and/or GSH in a leukemia cell by at least 30%, by at least 40%, by at least 50%, or by at least 60%.

4. (canceled)

5. The method according to claim 1, wherein the compound is a compound of Formula (I) and/or a isomers, stereoisomers or solvates thereof and/or mixtures thereof.

6. The method of claim 5, wherein the compound is a compound of Formula (I), optionally wherein the compound of Formula (I) has n=5.

7. (canceled)

8. The method of claim 6, wherein the compound comprises avocadyne, avocadyne acetate and/or avocadynone acetate or a mixture comprising avocadyne, avocadyne acetate and/or avocadynone acetate, optionally wherein the compound comprises avocadyne, optionally avocatin B and/or avocatin A.

9. (canceled)

10. The method of claim 1, wherein the method further comprises administering a chemotherapeutic selected from cytarabine and an anthracycline.

11. The method, wherein the leukemia is an acute myeloid leukemia (AML), an acute lymphoblastic leukemia (ALL), a chronic lymphocytic leukemia (CLL) or a chronic myelogenous leukemia (CML) or the leukemia cell is an AML cell, an ALL cell, a CLL cell or a CML cell.

12. A combination comprising chemotherapeutic, selected from cytarabine and an anthracycline, and a compound of Formula (I) and/or (II) having the structure: 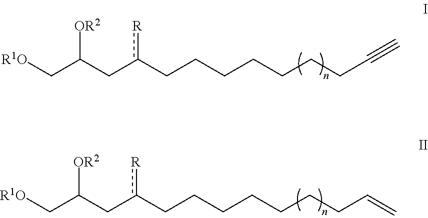
wherein: [symbol]
represents a single or a double bond;
R is OH when C [symbol] R is C—R, and R is O when C [symbol] R is C═R;
n is 1, 3, 5 or 7; and
R¹ and R² are independently hydrogen or acetyl,
or isomers, stereoisomers or solvates thereof and/or mixtures thereof, preferably wherein the compound is a compound of Formula (I), more preferably wherein the compound comprises a n=5, and even more wherein compound is avocadyne, avocadyne acetate and/or avocadynone acetate or a mixture comprising avocadyne, avocadyne acetate and/or avocadynone acetate.

13. The combination of claim 12, wherein the combination is for use in treating leukemia in a subject in need thereof.

14. The combination of claim 12, wherein the anthracycline is selected from daunorubicin, doxorubicin, mitoxantrone, idarubicin and amsacrine.

15.-20. (canceled)

21. The combination claim 12, wherein the compound of Formula (I) and/or (II) and/or isomers, stereoisomers or solvates thereof and/or mixtures thereof is selected from avocadene, avocadene acetate, avocadenone acetate, avocadyne, avocadyne acetate, avocadynone acetate, avocatin A and avocatin B and mixtures thereof.

22. The combination of claim 13, wherein the leukemia is an acute myeloid leukemia (AML), an acute lymphoblastic leukemia (ALL), a chronic lymphocytic leukemia (CLL) or a chronic myelogenous leukemia (CML) or the leukemia cell is an AML cell, an ALL cell, a CLL cell or a CML cell.

23. The method of claim 1, wherein the compound of Formula (I) and/or (II) and/or isomers, stereoisomers or solvates thereof and/or mixtures thereof s comprised in a composition, optionally a pharmaceutical composition.

24. The method of claim 23, wherein the pharmaceutical composition comprises a therapeutically effective amount of the compound of Formula (I) and/or (II) and/or isomers, stereoisomers or solvates thereof and/or mixtures thereof and one or more suitable excipients, diluents, buffers, carriers or vehicles.

25. The method of claim 23, wherein the pharmaceutical composition is in a dosage form selected from a solid dosage form and a liquid dosage form, optionally wherein the dosage form is selected from an oral and an injectable dosage form.

26. The method of claim 23, wherein the pharmaceutical composition is administered by parenteral, intravenous, subcutaneous, intramuscular, intraspinal, intracisternal, intraperitoneal, or oral administration.

27. (canceled)

28. (canceled)

29. A test assay for identifying for identifying putative combinations for treating leukemia comprising:
a. contacting a leukemia cell with a compound of Formula (I) and/or (II) having the structure: 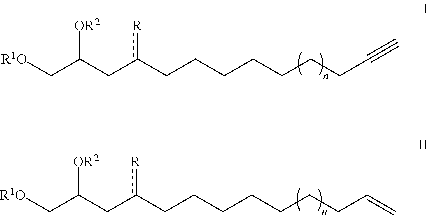
wherein: [symbol]
represents a single or a double bond;
R is OH when C [symbol] R is C—R, and R is O when C [symbol] R is C═R;
n is 1, 3, 5 or 7; and
R¹ and R² are independently hydrogen or acetyl,
and/isomers, stereoisomers or solvates thereof and/or mixture thereof in the presence and in the absence of a test agent;
b. measuring the viability of the cell in the presence of the test agent and in the absence of the test agent, optionally using a MTS assay;
c. determining if the compound of Formula (I) and/or (II) and/or isomers, stereoisomers or solvates thereof and/or mixtures thereof in combination with the test agent exhibit synergistic cytotoxicity; and
d. optionally testing synergistic combinations is a second viability assay.

30. The test assay of claim 29, wherein synergistic cytotoxicity is determined by measuring the combination index (CI), wherein CI<1 is indicative of a synergistic effect, CI=1 is indicative of an additive effect and CI>1 is indicative of an antagonistic effect, optionally wherein the test agent is a chemotherapeutic.

31. (canceled)

32. The test assay of claim 29, wherein the compound of Formula (I) and/or (II) and/or isomers, stereoisomers or solvates thereof and/or mixtures thereof in combination with the test agent increases mitochondrial fatty acid oxidation at least 2-fold compared to the compound of Formula (I) and/or (II) and/or isomers, stereoisomers or solvates thereof and/or mixture thereof in the absence of the test agent.

33. A method of identifying a subject with leukemia likely to benefit from administration of the combination of claim 12 or a compound of Formula (I) and/or (II) having the structure: 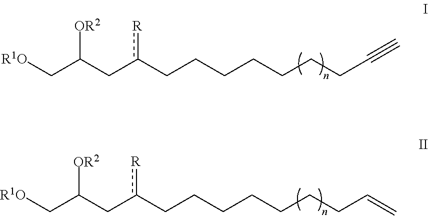
wherein: [symbol]
represents a single or a double bond;
R is OH when C [symbol] R is C—R, and R is O when C [symbol] R is C═R;
n is 1, 3, 5 or 7; and
R¹ and R² are independently hydrogen or acetyl,
and/or isomers, stereoisomers or solvates thereof and/or mixture thereof and optionally a chemotherapeutic, comprising:
a. obtaining a test sample comprising leukemia cells from the subject;
b. determining a mitochondrial mass of the test sample; and
c. comparing the mitochondrial mass of the test sample to a mitochondrial mass of a control;
wherein the subject is identified as likely to benefit from administration of the compound of Formula (I) and/or (II) and/or isomers, stereoisomers or solvates thereof and/or mixture thereof and optionally the chemotherapeutic when the leukemia cells have an at least 2 fold increased mitochondrial mass compared to the control.

34. A kit comprising the combination of claim 12 or a compound of Formula (I) and/or (II) having the structure: 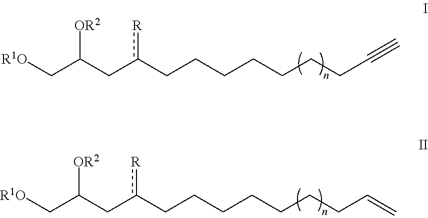
wherein: [symbol]
represents a single or a double bond;
R is OH when C [symbol] R is C—R, and R is O when C [symbol] R is C═R;
n is 1, 3, 5 or 7; and
R¹ and R² are independently hydrogen or acetyl,
and/or isomers, stereoisomers or solvates thereof and/or mixture thereof, optionally a chemotherapeutic, and/or packaging instructions for use thereof, and/or mixtures thereof, preferably wherein the compound is a compound of Formula (I), more preferably wherein the compound comprises a n=5, and even more wherein compound is avocadyne, avocadyne acetate and/or avocadynone acetate or a mixture comprising avocadyne, avocadyne acetate and/or avocadynone acetate.

35. The kit of claim 34, wherein the chemotherapeutic is selected from cytarabine and an anthracycline, optionally wherein the anthracycline is selected from daunorubicin, doxorubicin, mitoxantrone, idarubicin and amsacrine.

36. (canceled)
